(19) United States
(12) Patent Application Publication
(10) Pub. No.: US 2008/0318900 A1
(43) Pub. Date: Dec. 25, 2008

(54) Cosmetic Compositions Comprising At Least One Bis-Urea Derivative

(76) Inventor: Charlotte FELTIN, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/967,532

(22) Filed: Dec. 31, 2007

Related U.S. Application Data
(60) Provisional application No. 60/886,514, filed on Jan. 25, 2007.

(30) Foreign Application Priority Data
Dec. 29, 2006 (FR) 06/56062

(51) Int. Cl. A61K 8/06; A61K 8/40

(52) U.S. Cl. 514/63

(57) ABSTRACT

Disclosed herein is a cosmetic composition comprising at least one continuous liquid fatty phase, comprising at least one compound of formula (I), or a salt and/or isomer thereof:

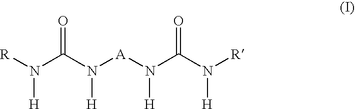

and at least one oil with a solubility parameter δₐ ranging from 0 to 7.00 (J/cm³)^{1/2}, for example from 0 and 5.00 (J/cm³)^{1/2}. The present disclosure also relates to a process for making up or caring for keratin materials, comprising applying to the keratin materials at least one composition as described above.

This application claims priority under 35 U.S.C. § 119 to U.S. Provisional Application No. 60/886,514, filed Jan. 25, 2007. This application also claims benefit of priority under 35 U.S.C. § 119 to French Patent Application No. 06/56062, filed Dec. 29, 2006.

The present disclosure relates to compositions for caring for or making up the skin of either the human face or body, keratin fibers, for instance the eyelashes, the eyebrows or the hair, or the lips.

Such compositions may be a foundation, a makeup rouge, an eyeshadow, a concealer, a lip cream, a mascara or a body makeup product, when it is in colored form, or alternatively may be a skincare cream, a hair conditioner, a shampoo, an antisun cream or skin-coloring cream or a dermatological ointment, when it is in uncolored form.

Care or makeup compositions may have varied textures ranging from fluid to solid. One of the difficulties encountered by users is that of being able to spread the composition uniformly over the entire surface of the face or body so as to distribute the product uniformly. Compositions of thick or solid texture are difficult to spread on account of their high viscosities. Compositions of fluid texture are not always suitable for obtaining a uniform makeup result, since they do not leave visible marks on the skin, for example on account of their poor spreading over the entire surface of the face to be made up. Creamy textures are thus often sought.

These creamy compositions generally contain a thickener that facilitates the uptake of the product from the packaging, without loss of product, allowing the product to the distributed homogeneously over the area to be treated or allowing sufficient amounts of product to be taken up, in order to obtain the desired cosmetic effect. When the composition is sufficiently thick, uptake of the product by finger produces hollows on the surface of the product in the packaging, and, the next time product is taken out, the surface of the product has remained as it was previously, for example at the time of the preceding closure of the jar; the cream then appears contaminated, giving rise to dissatisfaction on the part of the users.

For example, for a cream of rich and/or thickened texture, which does not flow, the absence of surface leveling of the cream may occur after each use.

There is thus still a need for compositions for making up, treating or caring for the skin and keratin fibers, which show good leveling of the surface in its packaging, giving the impression of opening a new jar on each occasion, and of touching an uncontaminated product.

The inventors have discovered, suprisingly, that such a composition is obtained by combining in the composition at least one bis-urea derivative and at least one oil.

One abspect of the present disclosure is a cosmetic composition comprising at least one continuous liquid fatty phase, comprising at least one compound chosen from compounds of formula (I), and salts and isomer thereof:

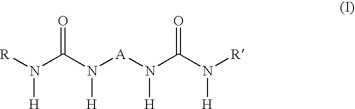

wherein:
A is a group of formula (II):

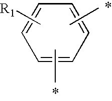

wherein R₁ being a linear or branched C₁-C₄ alkyl radical, and the asterisks, *, symbolize the points of attachment of the group A to each of the two nitrogen atoms of the rest of the compound of formula (I), and
R and R′, which may be identical or different, are chosen from:
i) the radicals of formula (III):

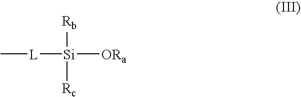

in which:
L is chosen from a single bond or a divalent carbon-based radicals, for example, linear, branched and cyclic, saturated and unsaturated hydrocarbon-based C₁-C₁₈ alkylene radical, optionally comprising 1 to 4 heteroatoms chosen from N, O and S;
Rₐ is:
   a) a carbon-based radicals, for example, a linear, branched and cyclic, saturated and unsaturated hydrocarbon-based C₁-C₁₈ alkyl radicals optionally comprising 1 to 8 heteroatoms chosen from N, O, Si and S; and
   b) a silicone radical of formula:

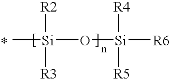

wherein n ranges from 0 and 100, for example, from 1 to 80, and for instance, 2 to 20;
and R₂ to R₆ are, independently of each other, chosen from carbon-based radicals, for example, linear or branched hydrocarbon-based alkyl radicals comprising 1 to 12 , for example, 1 to 6 carbon atoms, and optinally comprising 1 to 4 heteroatoms, such as O;
R_{b} and R_{c} are, independently of each other, chosen from:
a) carbon-based radicals, for example, linear, branched and cyclic, saturated and unsaturated hydrocarbon-based C₁-C₁₈ alkyl radicals optionaly comprising 1 to 4 heteroatoms chosen from N, O, Si and S;
b) the radicals of formula:

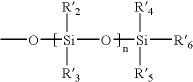

wherein n being between 0 and 100, for example, from 1 to 80, and for instance, 2 to 20;
and R′₂ to R′₆ are, independently of each other, chosen from carbon-based radicals, for example, linear or branched hydrocarbon-based alkyl radicals comprising 1 to 12, for example, 1 to 6 carbon atoms, and optionally comprising 1 to 4 heteroatoms, especially O; and
ii) linear, branched and/or cyclic, saturated and unsaturated C₁-C₃₀ alkyl radicals, optionally comprising 1 to 3 heteroatoms chosen from O, S, F and N;
   wherein at least one of the radicals R and R′ is f formula (III); and
   at least one oil with a solubility parameter δₐ ranging from 0 and 7.00 (J/cm³)^{1/2}, for example, from 0 to 5.00 (J/cm³)^{1/2}.

Another aspect of the present disclosure is a process for making up or caring for keratin materials, comprising applying the keratin materials at least one composition as defined above.

Yet another aspect of the present disclosure is the use of compositions as defined above for obtaining a makeup result that is smooth via leveling of the skin relief and/or matt and/or soft to the touch.

### Silicone Bis-Ureas

The compounds of bis-urea type as disclosed herein are chosen from those of formula (I) below:

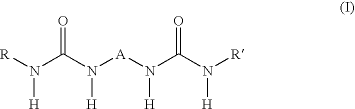

wherein:
A is chosen from groups of formula (II):

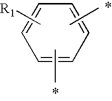

wherein R₁ is chosen from linear or branched C₁-C₄ alkyl radicals, and the asterisks, *, symbolizing the points of attachment of the group A to each of the two nitrogen atoms of the rest of the compound of formula (I), and
R and R′, which may be identical or different, are chosen from:
i) the radicals of formula (III):

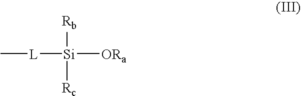

wherein:
L is a single bond or a divalent carbon-based radical, for example, a linear, branched and/or cyclic, saturated or unsaturated hydrocarbon-based C₁-C₁₈ alkylene radical, optionally comprising 1 to 4 heteroatoms chosen from N, O and S;
Rₐ is:
   a) a carbon-based radical, for example, a linear, branched and cyclic, saturated or unsaturated hydrocarbon-based C₁-C₁₈ alkyl radical comprising 1 to 8 heteroatoms chosen from N, O, Si and S; or
   b) a silicone radical of formula:

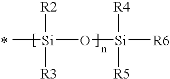

wherein n being between 0 to 100 ,for example, from 1 to 80 and for instance 2 to 20;
and R₂ to R₆ are, independently of each other, carbon-based radicals, for example, linear or branched hydrocarbon-based alkyl radicals comprising 1 to 12, for example, 1 to 6 carbon atoms, and optionally comprising 1 to 4 heteroatoms, such as O;
R_{b} and R_{c} are, independently of each other, chosen from:
   a) carbon-based radicals, for example, linear, branched and cyclic, saturated and unsaturated hydrocarbon-based C₁-C₁₈ alkyl radicals optionally comprising 1 to 4 heteroatoms chosen from N, O, Si and S;
   b) the radicals of formula:

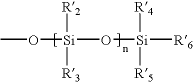

wherein n being between 0 to 100, for example, between 1 to 80, and for instance, 2 to 20;
and R′₂ to R′₆ are, independently of each other, carbon-based radicals, for example, linear or branched hydrocarbon-based alkyl radicals comprising 1 to 12, for example, 1 to 6 carbon atoms, and optionally comprising 1 to 4 heteroatoms, such as O; and
ii) linear, branched and cyclic, saturated and unsaturated C₁-C₃₀ alkyl radicals, optionally comprising 1 to 3 heteroatoms chosen from O, S, F and N;
   wherein at least one of the radicals R and R′ is of formula (III).

According to the present disclosure, when the two radicals R and R′ are for example, silicone-based, i.e. of formula (III), it has been found that this family of bis-urea compounds enables texturing of silicone media.

When at least one of the radicals R or R′ is non-silicone-based, i.e. is an alkyl radical as defined above, it is possible to texture with these compounds silicone media, as well as carbon-based media and media comprising a mixture of silicone oils and carbon-based oils.

According to at least one embodiment of the present disclosure, the group A may be of formula:

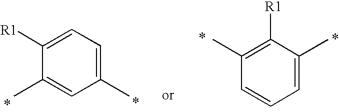

with R₁ and the asterisks, *, being as defined above.

For example, R₁ may be a methyl group, and group A may be of formula:

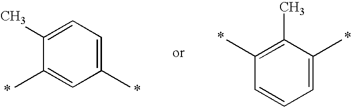

wherein the asterisks, *, are as defined above.

According to at least one embodiment of the present disclosure, A may be a mixture of 2,4-tolylene and 2,6-tolylene, for example, in (2,4 isomer)/(2,6 isomer) proportions ranging from 95/5 to 80/20.

As described herein, at least one of the radicals R and R′ should be of formula (III):

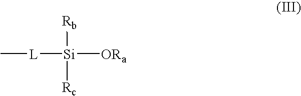

According to at least one embodiment of the present disclosure, L is divalent carbon-based radical, for example, a linear, branched and/or cyclic, saturated or unsaturated hydrocarbon-based C₁-C₁₈ alkylene radical, optionally comprising 1 to 4 heteroatoms chosen from N, O and S. In the radical L, the carbon-based chain may be interrupted with the at least one heteroatom and/or may comprise a substituent comprising the at least one heteroatom.

For example, L may have the structure —(CH₂)n- where n ranges from 1 to 18, for example, 2 to 12, and for instance, 3 to 8. In at least one embodiment of the present disclosure, L is chosen from methylene, ethylene, propylene and butylene radicals and for example, n-butylene or octylene.

The radical L may also be branched, for example of the type —CH₂—CH(CH₃)—, which leads to the radical of formula (III) below:

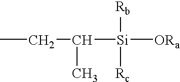

The radical Rₐ may be chosen from carbon-based radicals, for example, a linear, branched and cyclic, saturated or unsaturated hydrocarbon-based C₁-C₁₈ alkyl radicals optionally comprising 1 to 8 heteroatoms chosen from N, O, Si and S. The carbon-based chain may be interrupted with at least one heteroatom and/or may comprise a substituent comprising the at least one heteroatom; for example, the at least one heteroatom may form at least one —SiO— (or —OSi—) groups.

Thus, the radical Rₐ may have the structure —(CH₂)n′-CH₃ wherein n′ranges from 0 to 17, for example, 1 to 12 and for instance, from 1 to 6. For example, Rₐ may be chosen from methyl, ethyl, propyl or butyl.

It may also have the structure —(CH₂)x-O—(CH₂)z-CH₃ or —(CH₂)x-O—(CH₂)y-O—(CH₂)z-CH₃, wherein x ranges from 1 to 10, and for instance, is 2; y ranges from 1 to 10, and for instance, is 2, and z ranges from 0 to 10, and for instance, is chosen from 0 and 1.

The radical Rₐ may also have the structure —SiR₄R₅R₆ (in the case where n=0), wherein R₄, R₅ and R₆ are, independently of each other, chosen from for example, alkyl radicals containing 1 to 12 carbon atoms and for example, 1 to 6 carbon atoms; for example, R₄, R₅ and/or R₆ may be chosen from methyl, ethyl, propyl and butyl.

In at least one embodiment, the radical Rₐ may also be a silicone radical of formula:

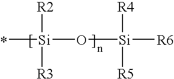

wherein R₂ to R₆ are, independently of each other, chosen from, for example, alkyl radicals comprising 1 to 12 carbon atoms and for instance, 1 to 6 carbon atoms; for example, R₂ to R₆ may be chosen from methyl, ethyl, propyl and butyl;
and in particular a radical:

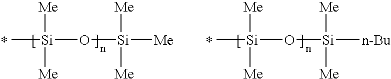

wherein n ranges from 1 to 100; and in at least one embodiment, a radical:

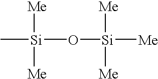

The radicals R_{b} and R_{c}, which may be identical or different, may be carbon-based radicals, chosen from, for example, linear, branched and cyclic, saturated or unsaturated hydrocarbon-based C₁-C₁₈ alkyl radicals optionally comprising 1 to 8 heteroatoms chosen from N, O, Si and S. In these radicals, the carbon-based chain may be interrupted with at least one heteroatom and/or may comprise a substituent comprising the at least one heteroatom; the at least one heteroatom may form, for example, at least one —SiO— (or —OSi—) group.

In at least one embodiment, they may have the structure —(CH₂)m-CH₃ wherein m ranges from 0 to 17, for example, 1 to 12, and for even instance, from 2 to 5; in at leats one embodiment, R_{b} and/or R_{c} may be methyl, ethyl, propyl and butyl.

They may also have the structure —O—(CH₂)m′-CH₃ wherein m′ranges from 0 to 5, for example, 1 to 4 such as methoxy or ethoxy.

They may also have the structure —O—(CH₂)x-O—(CH₂)z-CH₃ or —O—(CH₂)x-O—(CH₂)y-O—(CH₂)z-CH₃, wherein x ranges from 1 to 10, and for instance, is 2; y ranges from 1 to 10, and for instance, is 2, and z ranges from 0 to 10, and for instance, is chosen from 0 and 1.

They may also have the structure:

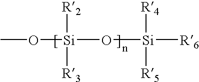

wherein n ranges from 0 and 100, for example, from 1 to 80 and for instance, 2 to 20;
and R′₂ to R′₆ being, independently of each other, are chosen from, for example, alkyl radicals comprising 1 to 12 carbon atoms and for instance 1 to 6 carbon atoms; in at least one embodiment, R′₂ to R′₆ may be chosen from methyl, ethyl, propyl and butyl.

When they are of formula (III), the radicals R and/or R′ are may be chosen, for example, chosen from the following radicals:

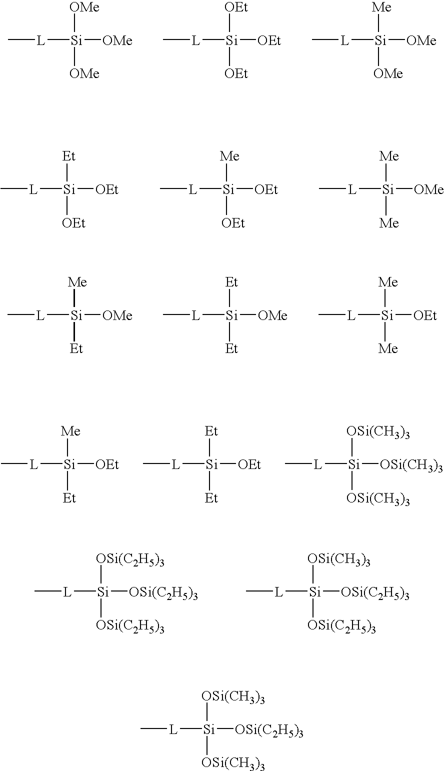

and also those of formulae:

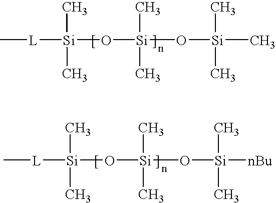

wherein n ranges from 0 to 100, and for instance

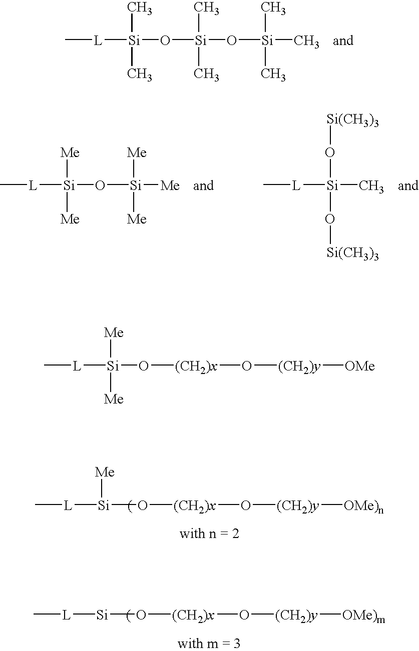

wherein x ranges from 1 to 10, and for instance is 2; and y ranges from 1 to 10, and for instance, is 2;
and L being as defined above.

In these formulae, L is, for example, chosen from linear and branched C₁-C₈ alkylene radical, such as methylene, ethylene, propylene and butylene and for example, n-butylene and octylene, and radicals of formula —CH₂—CH(CH₃)—.

In at least one embodiment, R and R′, which may be identical or different, are both of formula (III).

In another embodiment, one of the radicals R or R′ is chosen from linear, branched and/or cyclic, saturated or unsaturated C₁-C₃₀ alkyl radical, optionally comprising 1 to 3 heteroatoms chosen from O, S, F and N.

As disclosed herein, the compounds of formula (I) are of a universal nature, i.e. enabling them to texture, simultaneously, polar or apolar carbon-based media, linear or cyclic silicone media, and mixed oils, i.e. partially silicone-based carbon-based oils, and also mixtures thereof.

The carbon chain may be interrupted with the heteroatom with at least one heteroatom and/or may comprise a substituent comprising the said heteroatom, chosen from, for example, a carbonyl group (—CO—), hydroxyl radicals (—OH), and an ester radicals —COOR″ with R″=linear or branched alkyl radical containing 1 to 8 carbon atoms.

For example, the radical R or R′ may be a group chosen from:

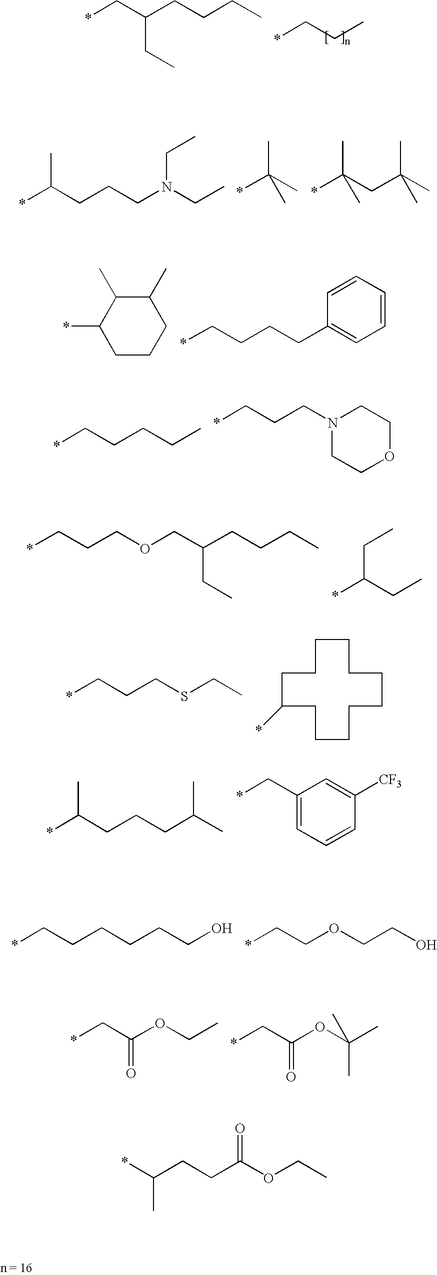

with the asterisks, *, having the definition given above.

In at least one embodiment, R or R′ represents a branched, for example, mono-branched, such as acyclic, saturated or unsaturated alkyl radical containing 3 to 16 carbon atoms, for example, 4 to 12, and for instance, 4 to 8 carbon atoms, and optionally comprising 1 to 3 heteroatoms chosen from O, S, F and N, for example, O and/or N.

For instance, R or R′ may be tert-butyl or 2-ethylhexyl radicals or of formula:

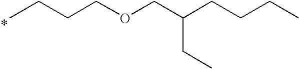

When the compound of formula (I) comprises a radical R that is an alkyl radical, and thus a radical R′ that is of formula (III), the ratio between n_{R} and n_{R′} may range, for example, between 5/95 to 95/5, for instance from 10/90 to 90/10, such as from 40/60 to 85/15, for example, from 50/50 to 80/20, and for instance, from 60/40 to 75/25;

with n_{R} being the number of moles of amine NH₂—R′ and n_{R′} being the number of moles of amine NH₂—R′ used to prepare the compound of formula (I).

The compounds disclosed herein may be in the form of salts and/or of isomers of compounds of formula (I).

For example, the compounds of formula (I) as disclosed herein may be chosen, alone or as a mixture, from the following compounds, and also the salts and isomers thereof:

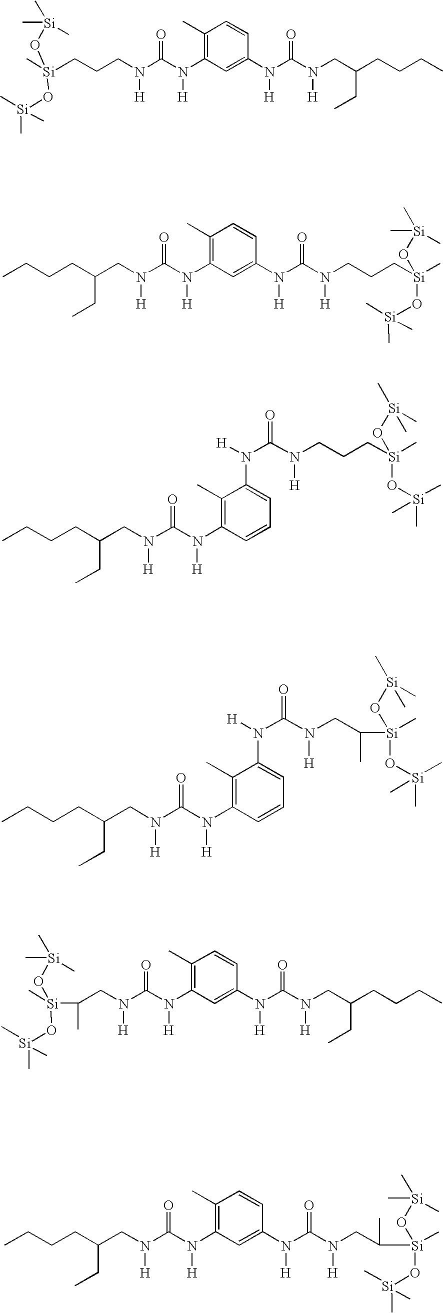

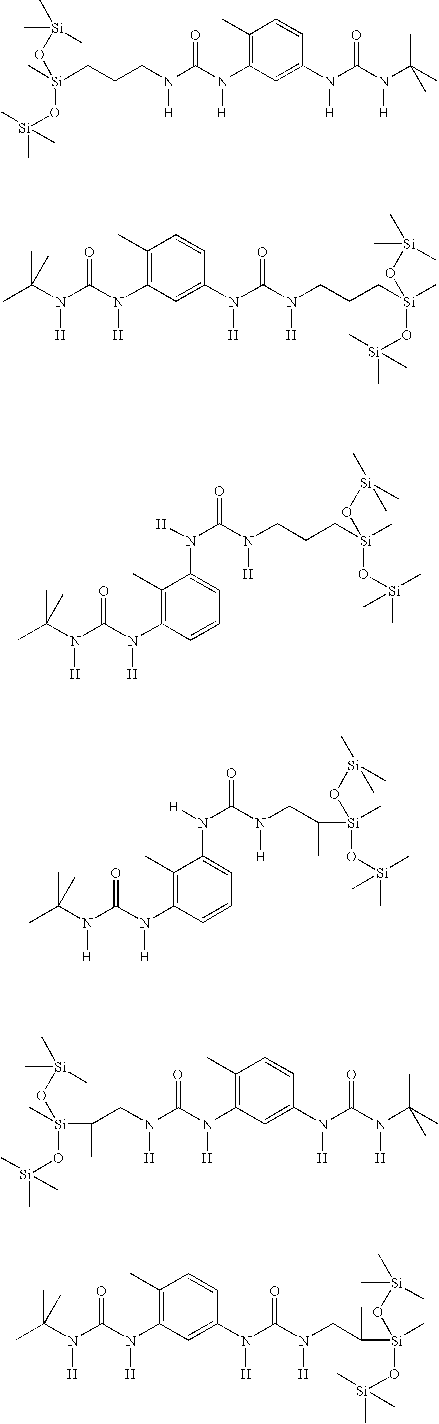

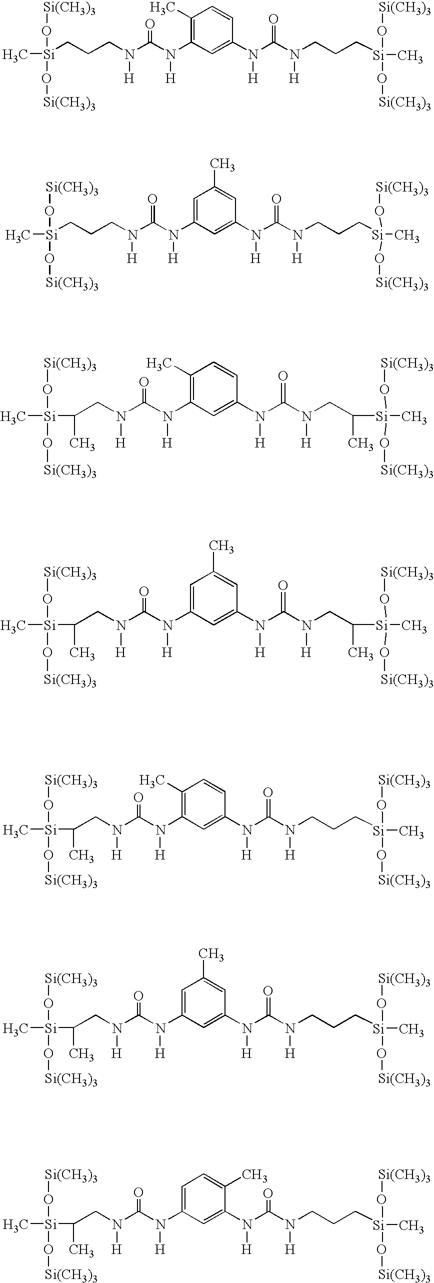

The compounds of formula (I) according to the present disclosure may be prepared by reaction between at least one diisocyanate of formula OCN-A-NCO, and at least one primary amine R—NH₂, with A and R as defined above.

When the radicals R and R′ are different, the diisocyanate may be reacted with a mixture of at least two primary amines: R—NH₂+R′—NH₂.

The diisocyanate OCN-A-NCO may be in the form of a mixture of positional isomers of the substituent R₁ on the group A, and for example, in 95/5 or 80/20 proportions (2,4-TDI isomer)/(2,6-TDI isomer).

In at least one embodiment, the amine(s) used is (are) in a mole ratio of 2 to 3 equivalents, for example, 2.1 to 2.5, such as 2.2 equivalents per one equivalent of the diisocyanate. The general reaction scheme is as follows:

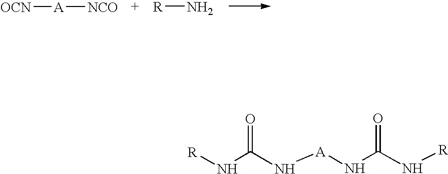

It is, quite obviously, possible to use a mixture of primary amines, for instance, two primary amines.

When two primary amines are used for the reaction, the mole ratio n_{R}/n_{R′} may range from 5/95 and 95/5; this ratio obviously depends on the chemical nature of each of the amines, and will be readily determined by a person skilled in the art on the basis of his knowledge.

This would be true, for example, when R and R′ are both of formula (III).

When R is an alkyl radical and thus R′ of formula (III), the said mole ratio n_{R}/n_{R′} may range from 5/95 to 95/5, for example from 15/85 and 90/10, for instance from 40/60 to 85/15 such as from 50/50 and 80/20;

with n_{R} being the number of moles of amine NH₂—R and n_{R′} being the number of moles of amine NH₂—R′ used to prepare the compound of formula (I).

Non-limiting mention may be made of the mixture of primary amines sold by Clariant under the name aminopropylbis(trimethylsiloxy)silane, which corresponds to a mixture comprising 80% to 99.5% by weight and for example 90% to 99% by weight of a first primary amine of formula NH₂—(CH₂)₃—Si[OSi(CH₃)₃]₂Me and 0.5% to 20% by weight and for instance 1% to 10% by weight of a second primary amine of formula NH₂—CH₂—CH(CH₃)—Si[OSi(CH₃)₃]₂Me.

The reaction can be performed under an inert atmosphere, for example under argon, in an anhydrous medium, with, for example, a reaction medium temperature that is maintained between 15° C. and 40° C.

The diisocyanate may be dissolved in anhydrous solvents such as tetrahydrofuran, 2-methyltetrahydrofuran, N-methylpyrrolidone, butyl acetate and methyl ethyl ketone at a concentration ranging from, for example, 1% to 30% by weight, for instance from 2% to 20% such as from 4% to 10% by weight. A solution comprising the amine(s) may be prepared in the same solvent as the diisocyanate, at a concentration that may range from 0.1% to 99.9% by weight.

The temperature of the reaction medium should, for instance, not exceed 40° C. and the concentration of the amine and the rate of addition of the solution comprising the amine may be adjusted to this requirement.

The reaction medium may be left stirring, for example for 30 minutes to 12 hours. The reaction progress may be monitored by infrared spectrometry (for instance by observing the disappearance of the NCO band between 2250 and 2280 cm⁻¹).

At the end of the reaction, the reaction medium may be poured into a large amount of acidified water (for example, of pH 3-4 with HCl). The precipitate, which is generally white, is then obtained, and is filtered off, washed, for example several times with for instance, water, and dried under reduced pressure, for example under vacuum or freeze-dried.

The precipitate corresponds to the expected compound of formula (I), or to the expected mixture of compounds of formula (I), and may be characterized by NMR spectrometry (¹H and/or ¹³C) and/or by HPLC coupled to mass spectrometry.

The compound may be used in its native form for the texturing of the oily medium under consideration.

For instance, the compounds of formula (I), alone or as a mixture in any proportion, can be dissolved in a wide variety of oils and thus allow for efficient texturing of the oils or the mixture of oils under consideration, giving it the desired physical and/or chemical properties.

In at least one embodiment, the compounds of formula (I) are soluble at a temperature of less than or equal to 50° C., or even less than or equal to 30° C., such as at room temperature (25° C.), in the liquid fatty phases usually used in cosmetics, and thus in the fatty phase to be textured.

The compounds disclosed herein thus are quite useful in the usual cosmetic or pharmaceutical compositions, for instance as an agent for texturing, thickening or even gelling the liquid fatty phase included in the said composition.

Said compounds of formula (I), alone or as a mixture, may be present in the compositions in an effective amount, i.e. in an amount that is necessary and sufficient to obtain the desired texturing of the liquid fatty phase under consideration in the composition according to the present disclosure.

As used herein, the expression “textured liquid fatty phase” means that the fatty phase takes the form of a gel or a thickened liquid. It can flow under its own weight. It can be deformed to constant volume if a stress is exerted thereon.

This texturing may be reflected by an increase in the viscosity which may be due to the introduction of at least one compound of formula (I).

Thus, the compositions according to the present disclosure may comprise from 0.01% to 20% by weight, for example, from 0.05% to 15% by weight, for instance, from 0.1% to 10% by weight, and for instance, from 1% to 8% by weight, such as from 2% to 5% by weight of at least one compound of formula (I) relative to the total weight of the composition.

The effective amount of the at least one compound of formula (I) may for example, represent from 0.01% to 20% by weight, for instance, from 0.05% to 15% by weight, for example, from 0.1% to 10% by weight, and for instance from 1% to 8% by weight such as from 2% to 5% by weight relative to the weight of the said liquid fatty phase.

This effective amount may vary depending, inter alia, on the nature of the bis-urea-based compound, on whether it is used in pure form or as a mixture with other bis-urea derivatives of formula (I), and on the nature of the liquid fatty phase.

### Hydrocarbon-Based Bis-Ureas

According to at least one particular embodiment of the present disclosure, the at least one compound of silicone bis-urea type described above may be mixed with at least one non-silicone bis-urea compound. According to a first aspect, the at least one non-silicone bis-urea compound may correspond to the formula (II) below:

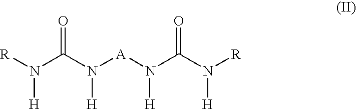

wherein:
A is a group of formula:

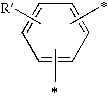

wherein R′ being a linear or branched C₁ to C₄ alkyl radical and the asterisks, *, symbolizing the points of attachment of the group A to each of the two nitrogen atoms of the rest of the compound of formula (II), and
R is chosen from saturated and unsaturated, non-cyclic, mono-branched C₆ to C₁₅ alkyl radicals whose hydrocarbon-based chain is optionally interrupted with 1 to 3 heteroatoms chosen from O, S and N, and
   a salts or isomers thereof.

According to at least one embodiment of the present disclosure, the group represented by A is a group of formula:

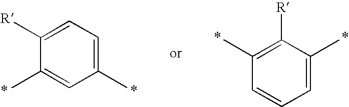

wherein R′ and the asterisks, *, are defined above.

For example, R′ may be a methyl group, and thus the group A may be a group of formula:

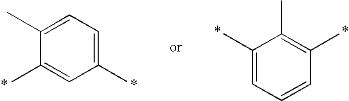

wherein the asterisks, *, are defined above.

According to one embodiment of the present disclosure, R may be chosen from the mono-branched radicals of general formula CₙH₂ₙ₊₁, wherein n is an integer ranging from 6 to 15, for example, from 7 to 9 such as 8.

Thus, the two groups R of the compound of formula (II) may represent, respectively, a group:

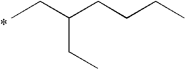

wherein the astrick, *, symbolizes the point of attachment of each of the groups R to each of the nitrogen atoms of the rest of the compound of formula (II).

According to another embodiment of the present disclosure, R may be chosen from the mono-branched radicals of formula Cₘ₋ₚH₂ₘ₊₁₋₂ₚXₚ, wherein p is equal to 1, 2 or 3, for instance, equal to 1, m is an integer ranging from 6 to 15, for example from 10 to 14, for instance from 10 to 12, such as 11, and X is chosen from S and O, for instance, oxygen atoms.

In at least one embodiment, R may be a radical of formula C_{m′}H_{2m′}X—(C_{p′}H_{2p′}X′)ᵣ—CₓH₂ₓ₊₁, wherein X and X′ are, independently of each other, chosen from S and O for instance, oxygen, r is 0 or 1, m′, p′ and x are integers such that their sum ranges from 6 to 15, for example, from 10 to 12, such as 11, and wherein at least one of the carbon-based chains C_{m′}H_{2m′}, C_{p′}H_{2p′}, or CₓH₂ₓ₊₁ is branched.

In at least one embodiment, wherein the chain CₓH₂ₓ₊₁ is branched, r is, for instance, equal to 0, m′ is for instance, an integer ranging from 1 to 10, for example from 2 to 6, such as 3, and/or x is, for example, an integer ranging from 4 to 16, for instance, from 6 to 12 such as 8.

Thus, the two groups R of the compound of formula (II) may each represent, independently, a group:

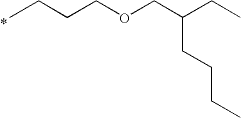

wherein the astrisk, *, symbolizes the point of attachment of each of the groups R to each of the nitrogen atoms of the rest of the compound of formula (II).

Such compounds may be present in the compositions according to the present disclosure as mixtures with isomers, for instance, positional isomers on the group A, for example, in 95/5 or 80/20 proportions.

As disclosed in the non-limiting examples below, and without wishing to be bound by theory, the presence of at least one of these radicals in the molecule of formula (II) proves to be beneficial for giving a universal nature, as disclosed herein, to the corresponding bis-urea derivatives.

Non-limiting mention may be made of the following compounds, used pure or as a mixture, as those which are suitable in ther context of the present disclosure:

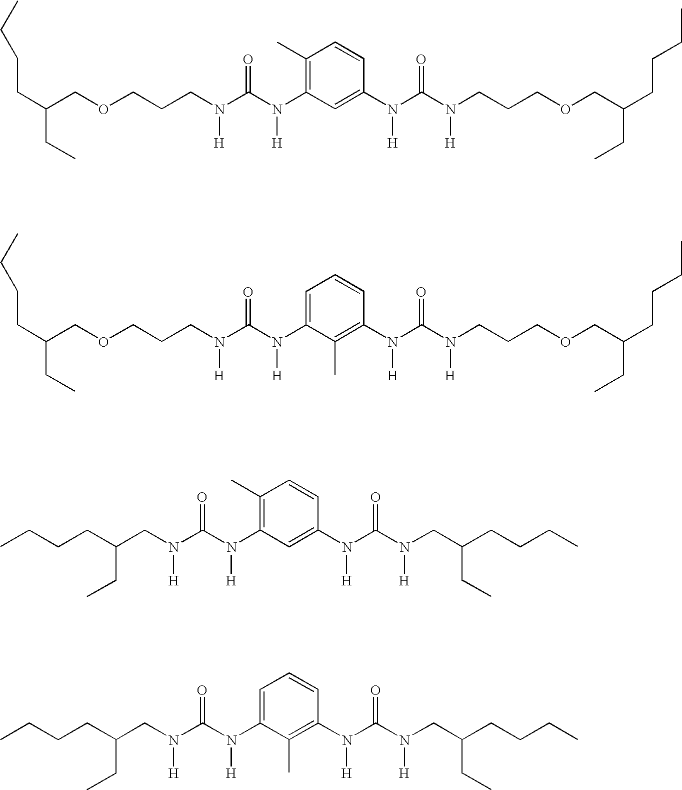

and the salts thereof.

As used herein, the term “suitable in the context of the present disclosure” means that the compound of formula (II), alone or as a mixture in all proportions, may be dissolved in a wide variety of oils and that it proves to be effective for texturing the at least one oil under consideration and thus may provide for it the desired physical and/or chemical properties.

As used herein, the term “physiologically acceptable” means a medium free of toxicity, which is compatible with application to the skin, the lips and/or the integuments of living beings, for example, human beings. Consequently, the compositions according to the present disclosure are free of compounds that are incompatible with and/or not tolerated for application to the skin, the lips and/or the integuments.

For the purposes of the present disclosure, the term “effective amount” means the amount that is necessary and sufficient to obtain texturing of the at least one oil under consideration in the composition according to the present disclosure.

As used herein, the term “textured liquid fatty phase”, means that the fatty phase takes the form of a gel or a thickened liquid. It may flow under its own weight. It may be deformed to constant volume if a stress is exerted thereon.

This texturing can be reflected by an increase in the viscosity which may be due to the introduction of at least one compound of formula (I).

For example, the compositions according to the present disclosure may contain from 0.01% to 20% by weight, for example, from 0.1% to 15% for instance from 1% to 10% such as from 2% to 8% by weight of the at least one compound of formula (II) relative to the total weight of the composition.

The effective amount of the at least one compound of formula (II) may represent from 0.01% to 20%, for example, from 0.05% to 10%, for example from 0.1% to 5% such as from 0.05% to 3% by weight of the liquid fatty phase.

This effective amount may vary significantly depending, partly on the nature of the substituent R of the bis-urea derivative, its position, whether or not it is used in pure form or as a mixture with other bis-urea derivatives of formula (II), and, partly the nature of the liquid fatty phase.

In general, the at least one compound of formula (II) according to the present disclosure is derived from the reaction between at least one diisocyanate of formula:

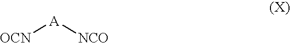

and a primary amine of formula:

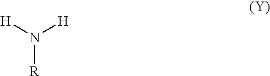

wherein A and R as defined above.

Where appropriate, the various diisocyanates (X) may be positional isomers of the substituent R′ on the group A, for example, in 95/5 or 80/20 proportions.

In at least one embodiment, the amine (Y) used is in a mole ratio of 2 to 3 equivalents, for example, 2.1 to 2.5 such as 2.2 equivalents per one equivalent of diisocyanate(s) (X). The general reaction scheme is as follows:

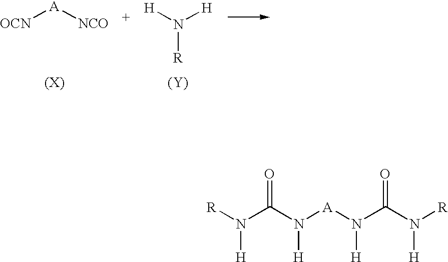

The reaction is generally performed under an inert atmosphere, for example under argon, in anhydrous medium with, for example, a reaction medium temperature that ranges from 15° C. to 40° C. and for instance, from 18° C. to 25° C.

The diisocyanate(s) (X) may be dissolved in an anhydrous solvent such as, but not limited to, tetrahydrofuran, 2-methyl-tetrahydrofuran, N-methylpyrrolidone, butyl acetate and methyl ethyl ketone, at a concentration which may range from 1% to 30% by weight, such as from 2% to 20% for example from 4% to 10% by weight.

A solution comprising the at least one amine of formula (Y) may be prepared in the same solvent as the diisocyanate of formula (X) at a concentration ranging, for example, from 0.1% to 99.9% by mass. The temperature of the reaction medium should for instance, not exceed 40° C. and the concentration of the amine and the rate of addition of the solution comprising the at least one amine of formula (Y) may be adjusted to this necessity. The reaction medium may be left stirring, for example, for 30 minutes to 12 hours. Monitoring of the reaction progress may be performed by infrared spectrometry (for instance, by observing the disappearance of the NCO band between 2250 and 2280 cm⁻¹). For example, at the end of the reaction, the reaction medium may be poured into a large amount of acidified water (for example, at pH 3-4 with HCl). A precipitate is then obtained, which is filtered off, washed, for example several times for example, with water, and dried under reduced pressure, for example, under vacuum or freeze-dried. The precipitate corresponds to the expected compounds of formula (II), and may be characterized by NMR spectrometry (¹H and/or ¹³C) and/or by HPLC and may be used as obtained for the texturing of the oily medium under consideration.

In one embodiment of the present disclosure, the bis-urea or the bis-urea is soluble at a temperature of less than or equal to 50° C., or even less than or equal to 30° C., and for example, at room temperature, in the liquid fatty phase to be textured.

According to another aspect of the present disclosure, the non-silicone bis-urea derivatives are chosen from compounds of of formula (III) below:

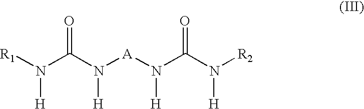

wherein:
A is a group of formula:

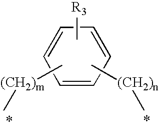

wherein
R₃ is chosen from a hydrogen atom and a linear and branched C₁ to C₄ alkyl radicals,
n and m, independently of each other, are equal to 0 or 1, and
the asterisk, *, symbolizes the point of attachment of the group A to the two nitrogen atoms of the residue of the compound of formula (III),
   R₁ is chosen from saturated and unsaturated, non-cyclic branched C₃ to C₁₅ carbon-based radicals optionally comprising from 1 to 3 heteroatoms chosen from O, S, F and N and/or a carbonyl,
R₂ is different from R₁ and is chosen from linear, branched or cyclic, saturated and unsaturated C₁-C₂₄ alkyl radicals optionally comprising from 1 to 3 heteroatoms chosen from O, S, F and N, and optionally substituted with:
   1, 2 or 3 hydroxyl radicals,
   an ester radical (—COOR₄), with R₄ is chosen from linear and branched alkyl radicals comprising from 1 to 8, for example, 1 to 6 , for instance 2 to 4 carbon atoms;
   saturated, unsaturated and aromatic C₅-C₁₂ cyclic radicals, such as a phenyl radical optionally substituted with at least one radical, which may be identical or different, chosen from C₁-C₄ alkyl and trifluoromethyl radicals, or morpholine derivative, and/or
   at least one radical chosen from linear and branched C₁-C₄ alkyl radicals, and salts or isomers thereof.

In at least one embodiment, n and m are equal, and may be equal to zero, and R₃ is a radical R′₃ as defined below. Thus, in one embodiment for example, A is a group:

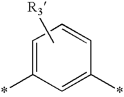

wherein R₃′ is chosen from linear and branched C₁ to C₄ alkyl radicals and the asterisks, *, symbolize the points of attachment of the group A to the two nitrogen atoms of the residue of the compound of formula (III).

According to one embodiment of the present disclosure, the compound of formula (III) comprises, as A, at least one group chosen from:

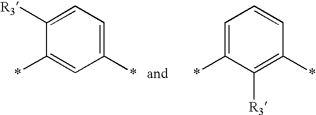

wherein R_{3′} and the asterisks, *, are defined above.

For example, R₃′ may be a methyl group, and thus the group A may be chosen from the groups:

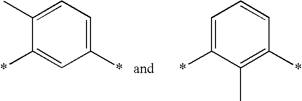

wherein the astericks, *, are defined above.

For example, the compounds can be such that A is a mixture of 2,4-tolylene and 2,6-tolylene, for example, in (2,4 isomer)/(2,6 isomer) proportions ranging from 95/5 to 80/20.

According to one embodiment of the present disclosure, the compound of formula (III) comprises, as R₁, a branched C₆-C₁₅ radical.

According to one embodiment of the present disclosure, the compound of formula (III) comprises, as R₁, a group chosen from:

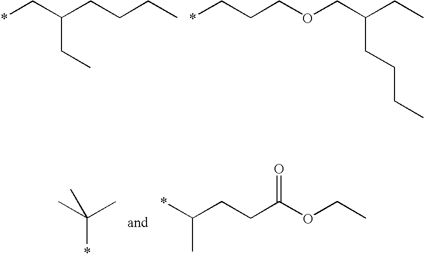

wherein the asterisks, *, symbolizes the point of attachment of the group R₁ to the nitrogen of the residue of the compound of formula (III).

As disclosed in the non-limiting examples below, the presence of at least one the two radicals in the molecule of formula (III) may give a universal nature within the meaning of the present disclosure to the corresponding asymmetric bis-urea derivatives.

In at least one embodiment, R₂, which may be different from R₁, may be chosen from the following groups:

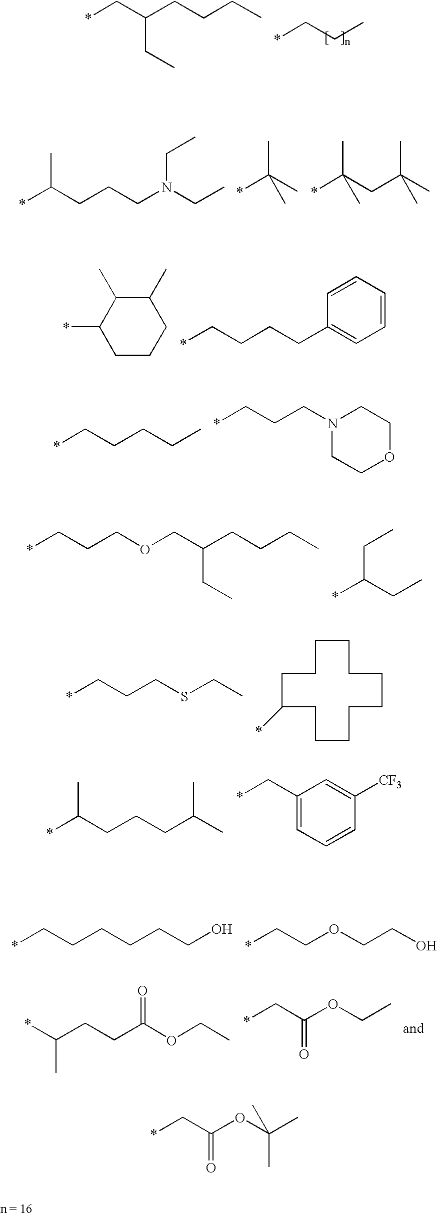

wherein the asterisk, *, symbolizes the point of attachment of the group R₂ to the nitrogen of the residue of the compound of formula (III).

Mention may be made of the following compounds as non-limiting illustrations of compounds that are suitable in the context of the present disclosure:

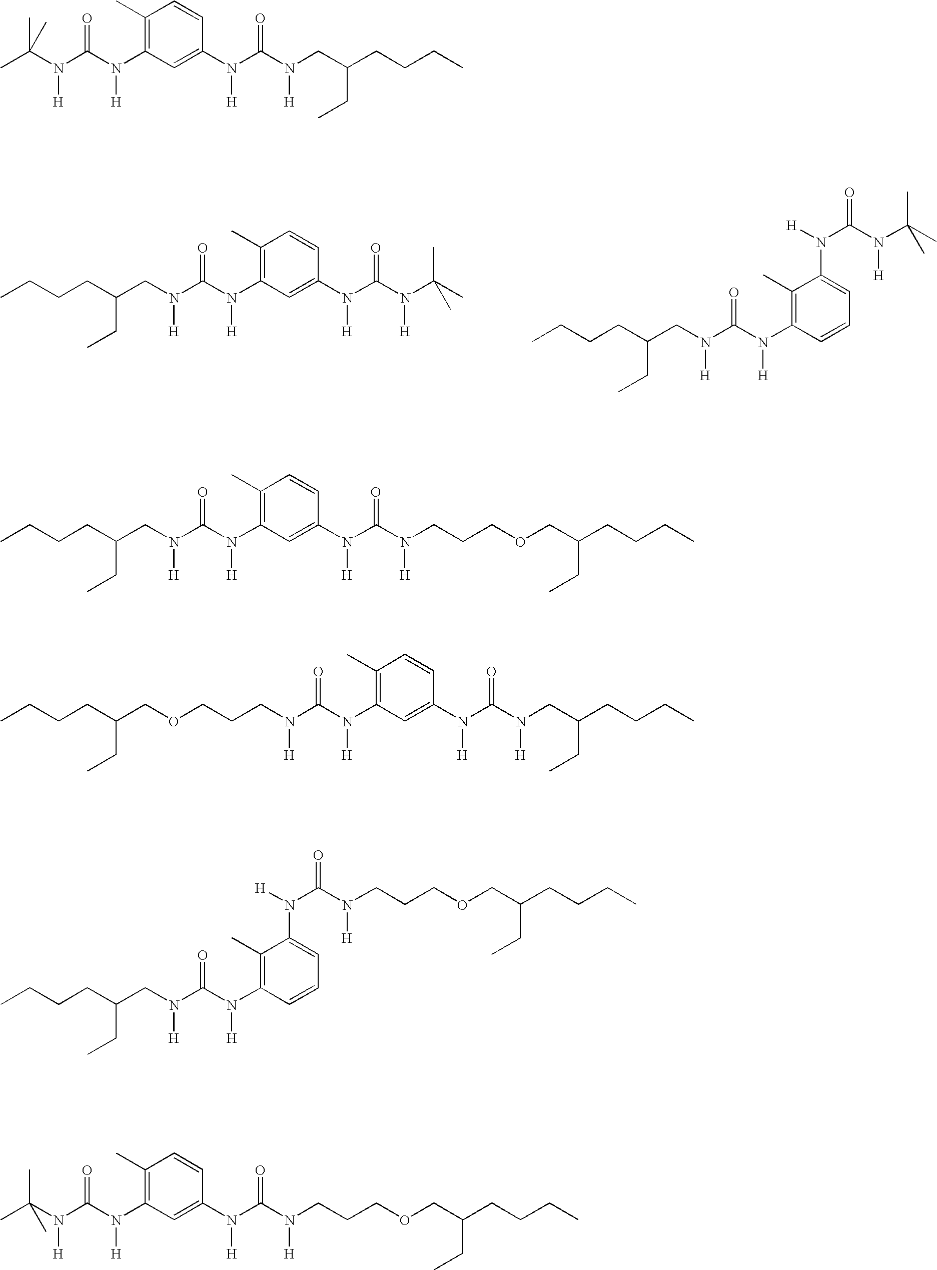

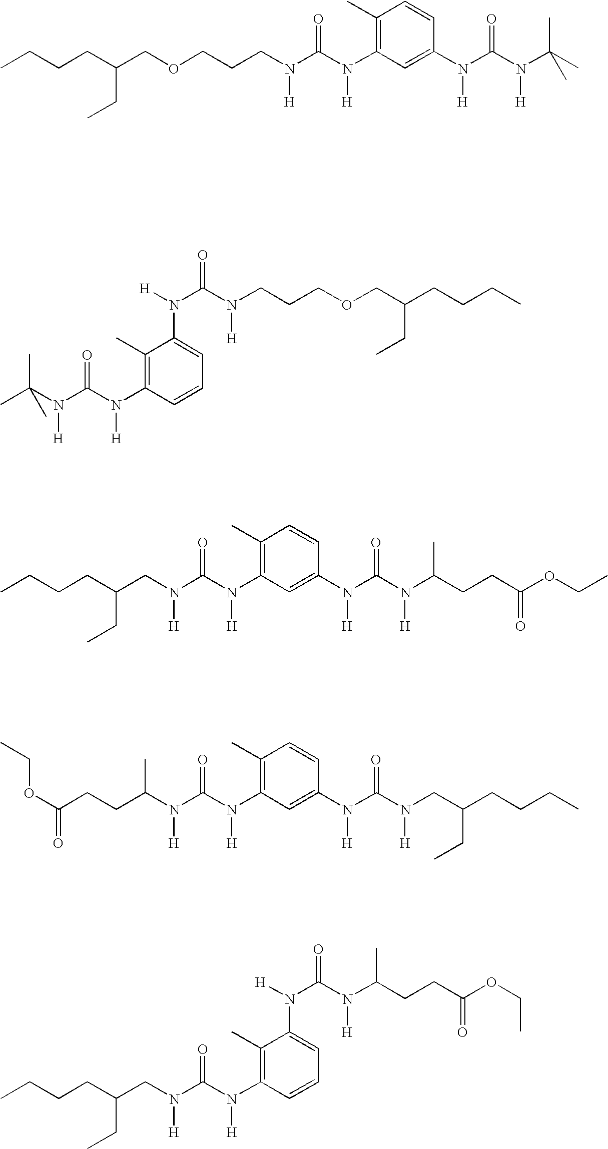

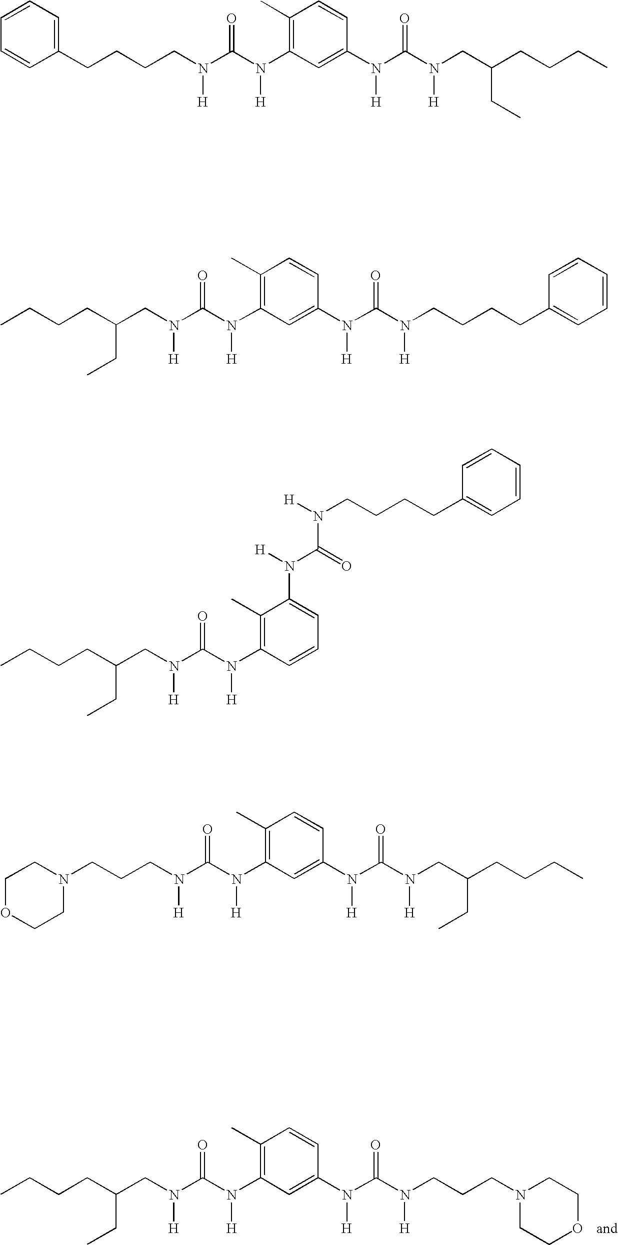

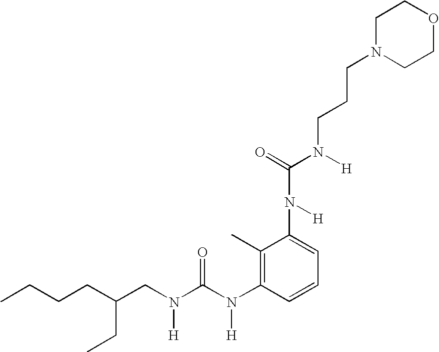

As used herein, the expression “suitable in the context of the present disclosure” refers to the compound of formula (III), alone or as a mixture in various proportions, which can dissolve at room temperature in several cosmetic oils and can be effective for gelling the at least one oil mixture under consideration and thus giving it the desired physical and/or chemical properties.

For the purposes of the present disclosure, the term “effective amount” means the amount that is necessary and sufficient to obtain texturing of the at least one oil under consideration in the composition according to the present disclosure.

This texturing can be reflected by an increase in the viscosity, which may be due to the introduction of at least one compound of formula (III).

For example, the compositions according to the present disclosure may contain from 0.0001% to 5% by weight of asymmetric bis-urea, for example, from 0.001% to 1% for instance from 0.004% to 0.5% by weight of at least one asymmetric bis-urea relative to the total weight of the liquid fatty phase.

This effective amount may vary significantly depending partly on the nature of the substituents R₁ and/or R₂ of the bis-urea derivative, the positional isomer, and whether or not it is used in pure form or as a mixture with other bis-urea derivatives of formula (III), and partly on the nature of the oily phase.

The asymmetric bis-urea derivatives of formula (III) may be used in the context of the present disclosure in the form of a mixture of derivatives of formula (III) with each other and/or with the two corresponding forms of symmetric bis-urea derivatives. For the purposes of the present disclosure, the term “symmetric bis-ureas” means the bis-ureas according to formula (III) wherein the radicals R₁ and R₂are identical.

In general, the compounds of formula (III) according to the present disclosure are derived from the reaction between at least one diisocyanate of formula:

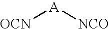

and at least two different primary amines of formulae:

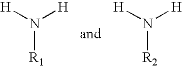

wherein A, R₁ and R₂ are defined as above.

Where appropriate, the various diisocyanates may be positional isomers of the substituent R₃ on the group A, for example, in 95/5 or 80/20 proportions.

As used herein, the term “at least two different primary amines” means that other primary amines Rₓ—NH₂ in which Rₓ corresponds to the definitions proposed for R₁ and R₂, may be added thereto.

The number of amines used for the reaction may be greater than or equal to 2 and may range, for example, from 2 to 20 and for instance from 3 to 10. In one embodiment, two amines may be used in order to facilitate the preparation of the composition and the characterization of the mixture thus obtained.

For instance, the amines used are, as a whole, in a mole ratio of 2 to 3 equivalents, for example, 2.1 to 2.5, such as 2.2 equivalents, per one equivalent of diisocyanate(s).

Where only two primary amines are used for the reaction, the mole ratio n(R₁)/n(R₂) may range from 1/99 to 99/1, for example from 5/95 to 95/5, for instance from 10/90 to 90/10, wherein n(R₁) corresponding to the number of moles of:

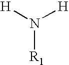

and n(R₂) corresponding to the number of moles of:

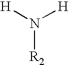

and with R₁ and R₂ are defined as above.

The reaction may be performed under an inert atmosphere, for example under argon, in an anhydrous medium with, for example, a reaction medium temperature maintained below 50° C., such as from 15° C. to 40° C., for example, from 18° C. and 25° C.

The at least one diisocyanate may be dissolved in at least one anhydrous solvent such as, but not limited to, tetrahydrofuran, 2-methyltetrahydrofuran, N-methylpyrrolidone, butyl acetate or methyl ethyl ketone to a concentration that may range from 1% to 30% by weight, for example, from 2% to 20%, such as from 4% to 10% by weight.

A solution comprising the amines is generally prepared in the same solvent as the at least one diisocyanate to a concentration ranging, for example, from 0.1% to 99.9% by mass. The temperature of the reaction medium should not exceed, for instance, 40° C. and the amine concentration and the rate of addition of the solution comprising the amines may be adjusted to this need. The reaction medium may be left stirring, for example, for 30 minutes to 12 hours. The reaction progress may be monitored by infrared spectrometry (for example, by observing the disappearance of the NCO band between 2250 and 2280 cm⁻¹). For example, at the end of the reaction, the reaction medium is poured into a large amount of acidic water (for example, of pH 3-4 with HCl). A precipitate is then obtained, which is filtered off, washed, for example several times for example, with water, and dried under reduced pressure, for example, under vacuum or freeze-dried.

After this reaction, at least one of the following two compounds forms:

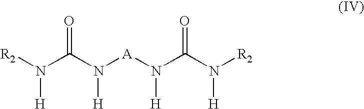

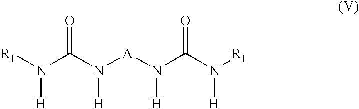

wherein A, R₁ and R₂ as defined above, with A being identical in formulae (III), (IV) and (V), R₁ being identical in formulae (III) and (V) and R₂ being identical in formulae (III) and (IV),
can be obtained together with the expected asymmetric derivative of formula (III).

The precipitate corresponds to the expected compounds of formula (III), and may be characterized by NMR spectrometry (¹H and/or ¹³C) and/or by HPLC and may be used in its native form for texturing the oily medium under consideration.

At the end of the reaction, the mixture of bis-ureas (III), (IV) and (V) is isolated and may be isolated and may be used in its native form for gelling the desired oily medium.

In this case, the at least one compound of formula (III) may be used in the liquid fatty phase in the form of a mixture with the at least one compound of formula (IV) and the at least one compound of formula (V).

In at least one embodiment, the mixture of bis-ureas is soluble at a temperature of less than or equal to 50° C., such as less than or equal to 30° C., and for example, at room temperature, in the liquid fatty phase to be textured.

### Silicone Bis-Urea Compatible Oils

The composition according to the present disclosure comprises at least one liquid fatty phase comprising at least one oil with a solubility parameter δₐ ranging from 0 to 7.00 (J/cm³)^{1/2} for example,from 0 and 5.00 (J/cm³)^{1/2}.

The solubility parameter δₐ is calculated by the relationship δₐ=(δ_{P}²+δₕ²)^{1/2} in which the parameters δ_{P} and δₕ correspond to the Hansen solubility parameters:
δ_{P} characterizes the Debye forces of interaction between permanent dipoles;
δₕ characterizes specific interaction forces (such as hydrogen bonding, acid/base, donor/acceptor, etc.).

The parameters δ_{P} and δₕ are generally expressed in (J/cm³)^{1/2}. They are determined at room temperature (25° C.) and in particular according to the calculation method indicated in patent JP-A-08-109 121.

The definition of the Hansen solubility parameters is well known to those skilled in the art, and is for example, described in the article by C. M. Hansen: “The three-dimensional solubility parameters”, J. Paint Technol., 39, 105 (1967). These parameters are also described in the Kao document JP-A-08-109 121 and the D. W. Van Krevelen document “Properties of polymers” (1990), p. 190.

The at least one oil with a solubility parameter δₐ of between 0 and 7.00 (J/cm³)^{1/2} may be chosen from hydrocarbon-based oils and silicone oils.

The at least one oil with a solubility parameter δₐ of between 0 and 7.00 (J/cm³)^{1/2} may be chosen for example, from squalane, polyisobutylenes with a molecular weight of between 250 and 800 g/mol, such as parleam oil, jojoba oil, arachidyl propionate, octyldodecyl stearoylstearate, arara oil, PVP/hexadecene copolymer, oleyl erucate, octyldodecyl stearate, isostearyl palmitate, isocetyl stearate, didecene, hydrogenated polydecene, isostearyl isostearate, dicaprylyl ether, isoeicosane, dioctyldodecyl dimer dilinoleate, octyldodecyl myristate, triisostearyl trilinoleate, octyldodecyl neodecanoate, octyldodecyl octanoate, isostearyl isononanoate, isofol-24 isostearate, octyldodecyl erucate, sesame oil, isopropyl myristate, linear silicone oils such as polydimethylsiloxanes, isobutyl stearate, isopropyl palmitate, stearyl heptanoate, isopropyl stearate, caprylic/capric triglycerides, propylene glycol dipelargonate, cyclopentadimethylsiloxane, cyclotetradimethylsiloxane, isostearyl neopentanoate, 2-ethylhexyl palmitate, diethylhexyl adipate, isopropyl isostearate, C₁₂-C₁₅ alkyl benzoate, pentaerythrityl tetrakis(2-ethylhexanoate), macadamia oil, isodecyl neopentanoate, phenyl trimethicone, propylene glycol dioctanoate, 2-ethylhexyl 2-ethylhexanoate, diisopropyl dimer dilinoleate, cetyl 2-ethylhexanoate, dicaprylyl maleate, isononyl isononanoate, cyclohexadimethylsiloxane, triisocetyl citrate, diisocetyl dodecanedioate, triisoarachidyl citrate, triisostearyl citrate, octyldodecyl neopentanoate, glyceryl triisononanoate, penta-erythrityl tetraisostearate, tridecyl trimellitate, glyceryl triisostearate, neopentyl glycol diheptanoate, tricapryline, polyglyceryl-2 triisostearate, tridecyltetradecanoin, dilinoleyl diol dimer/dilinoleic dimer copolyol, caprylyl carbonate, hexyldecyl myristoyl methylaminopropionate, polyglyceryl-10 nonaisostearate, dipentaerythrityl hexacaprylate/hexacaprate, the hydrogenated copolymer of dilinoleyl dimer/dimethyl carbonate, neopentyl glycol dicaprate, glyceryl trioctanoate, 2-ethylhexyl isostearate, diethylene glycol diisononanoate, undecylpentadecanol, isostearyl benzoate, isodecyl isononanoate, diisostearyl adipate, tridecyl isononanoate, triisopalmitine, 2-octyldodecyl hydroxystearate, butyl isostearate, 2-hexyldecyl isononanoate, 2-octyldodecyl benzoate, diisoarachidyl dodecanedioate, propylene glycol diisostearate, pentaerythrityl tetraisononanoate, pentaerythrityl tetrapelargonate, pentaerythrityl tetraoctyldodecanoate, pentaerythrityl tetradecyltetradecanoate, polyglyceryl-2 tetraisostearate, triisodecyl trimellitate, tris(2-ethylhexyl) trimellitate, isofol-12 trimellitate, pentaerythrityl tetrakis(2-hexyldecanoate), oxypropylenated (3 PO) myristyl diadipate, 2-ethylhexyl benzoate, ditrimethylolpropane tetraisostearate, castor oil benzoate, dioctyl (2-ethylhexyl) carbonate, octyldodecyl PPG-3 myristyl ether dimer dilinoleate, trimethylolpropane triisostearate, sucrose comprising 6-8 soybean fatty chains, and sesame oil, and mixtures thereof.

According to one aspect of the present disclosure, the at least one oil with a solubility parameter δₐ of between 0 and 7.00 (J/cm³)^{1/2} is a hydrocarbon-based oil chosen from aprotic alkanes.

According to a more preferred mode, the aprotic alkanes have a molecular mass ranging from 165 to 900 g/mol for example, from 250 to 800 g/mol.

In one embodiment of the present disclosure, the aprotic alkanes may be chosen, for example, from polyisobutylenes with a molecular weight ranging from 250 to 800 g/mol, squalane and parleam oil, and mixtures thereof.

According to another aspect of the present disclosure, the at least one oil with a solubility parameter δₐ of between 0 to 7.00 (J/cm³)^{1/2} is a silicone oil.

In at least one embodiment, the at least one silicone oils with a solubility parameter δₐ of between 0 and 7.00 (J/cm³)^{1/2} may be chosen from, for example, linear silicone oils such as polydimethylsiloxanes, cyclopentadimethylsiloxane, cyclotetradimethylsiloxane, phenyl trimethicone and cyclohexadimethylsiloxane, and mixtures thereof.

In one embodiment, the silicone is branched with groups of low molecular weight, for example, of C₁-C₃, such as methyl or ethyl groups.

According to one embodiment of the present disclosure, the at least one silicone oil with a solubility parameter δₐ of between 0 and 7.00 (J/cm³)^{1/2} has a molecular weight of between 100 and 800 g/mol, for example, from 300 to 500 g/mol.

The at least one oil with a solubility parameter δₐ of between 0 and 7.00 (J/cm³)^{1/2} may be present in the composition according to the present disclosure in an amount ranging from 20% to 100% by weight, for example, from 40% to 99% by weight, for instance, from 60% to 95% by weight relative to the total weight of the continuous liquid fatty phase.

### Additional Oils:

In addition to the at least one oil defined above, compositions according to the present disclosure may comprise at least one additional oil chosen from volatile oils and non-volatile oils.

According to at least embodiment, the composition according to the present disclosure may comprise at least one volatile oil.

As disclosed herein, the term “volatile oil” means any oil that is capable of evaporating on contact with the skin, at room temperature and atmospheric pressure. The at least one volatile oil of the present disclosure may be chosen from volatile cosmetic oils that are liquid at room temperature, with a non-zero vapour pressure at room temperature and atmospheric pressure, ranging for instance, from 0.13 Pa to 40 000 Pa (0.001 to 300 mmHg) and for example, ranging from 1.3 Pa to 1300 Pa (0.01 to 10 mmHg).

The at least one volatile oil may be chosen from volatile hydrocarbon-based oils, volatile silicone oils and volatile fluoro oils.

According to one preferred embodiment, the composition according to the present disclosure may comprise at least one hydrocarbon-based volatile oil.

As used herein, the term “hydrocarbon-based oil” means an oil mainly comprising hydrogen and carbon atoms and possibly oxygen, nitrogen, sulfur and/or phosphorus atoms.

In one embodiment, the at least one volatile hydrocarbon-based oils may be chosen from hydrocarbon-based oils comprising from 8 to 16 carbon atoms, such as branched C₈-C₁₆ alkanes, for instance C₈-C₁₆ isoalkanes of petroleum origin (also known as isoparaffins), for instance isododecane (also known as 2,2,4,4,6-pentamethylheptane), isohexadecane and, for example, the oils sold under the trade names Isopar® and Permethyl®.

Volatile oils that may also be used include volatile silicones, for instance volatile linear or cyclic silicone oils, for example, those with a viscosity ≦5 centistokes (5 ×10⁻⁶ m²/s) and, for example, those comprising from 2 to 10 silicon atoms, for instance, from 2 to 7 silicon atoms, wherein these silicones optionally comprise C₁-C₁₀ alkyl or alkoxy groups. As volatile silicone oils that may be used according to the present disclosure, non-limiting mention may be made of, for example, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

In one embodiment, the at least one volatile fluoro oil generally does not have a flash point.

Non-limiting mention may be made of volatile fluoro oils such as nonafluoroethoxybutane, nonafluoromethoxybutane, decafluoropentane, tetradecafluorohexane and dodecafluoropentane, and mixtures thereof.

The composition according to the present disclosure may comprise at least one volatile oil in a amount ranging from 1% to 50% by weight, for example, from 2% to 30% by weight, for instance, from 3% to 15% by weight, relative to the total weight of the composition.

The composition according to the present disclosure may comprise at least one non-volatile oil.

As used herein, the term “non-volatile oil” means an oil that remains on the skin at room temperature and atmospheric pressure for at least several hours and, for example, has a vapour pressure of less than 0.13 Pa (0.01 mmHg).

The at least one non-volatile oil may be chosen from hydrocarbon-based oils, for example, of animal or plant origin, or silicone oils, and mixtures thereof. As used herein, the term “hydrocarbon-based oil” means an oil mainly comprising hydrogen and carbon atoms and possibly oxygen, nitrogen, sulfur and/or phosphorus atoms.

The at least one non-volatile oil may be chosen from, for example, hydrocarbon-based oils, which may be fluorinated, and non-volatile silicone oils.

Non-limiting mention may be made of non-volatile hydrocarbon-based oils including, for example,
hydrocarbon-based oils of animal origin,
hydrocarbon-based oils of plant origin, such as triglycerides consisting of fatty acid esters of glycerol, wherein the fatty acids of which may have varied chain lengths from C₄ to C₂₄, and wherein these chains may be linear or branched, and saturated or unsaturated; such oils include, but not limited to, for example, heptanoic or octanoic acid triglycerides, wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, sesame seed oil, marrow oil, rapeseed oil, blackcurrant oil, evening primrose oil, millet oil, barley oil, quinoa oil, rye oil, safflower oil, candlenut oil, passionflower oil or musk rose oil; shea butter; or caprylic/capric acid triglycerides, for instance those sold by the company Stéarineries Dubois and those sold under the names Miglyol 810®, 812® and 818® by the company Dynamit Nobel;
synthetic ethers comprising from 10 to 40 carbon atoms,
linear or branched hydrocarbons of mineral or synthetic origin, such as petroleum jelly, polydecenes, hydrogenated polyisobutene such as Parleam®, squalane, liquid paraffins, and mixtures thereof;
synthetic esters, for instance oils of formula R₁COOR₂ in which R₁ is chosen from linear and branched fatty acid residue comprising from 1 to 40 carbon atoms and R₂ is chosen from hydrocarbon-based chains, which may be branched, and comprise from 1 to 40 carbon atoms, such that the number of carbon atoms in R₁+R₂≧10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, isodecyl neopentanoate, 2-ethylhexyl palmitate, isostearyl isostearate, 2-hexyldecyl laurate, 2-octyldecyl palmitate, 2-octyldodecyl myristate, or alcohol or polyalcohol heptanoates, octanoates, decanoates or ricinoleates, for instance propylene glycol dioctanoate; hydroxylated esters, for instance isostearyl lactate, diisostearyl malate or 2-octyldodecyl lactate; polyol esters and pentaerythritol esters;
fatty alcohols that are liquid at room temperature with a branched and/or unsaturated carbon-based chain comprising from 12 to 26 carbon atoms, for instance octyldodecanol, isostearyl alcohol, oleyl alcohol, 2-hexyldecanol, 2-butyloctanol or 2-undecylpentadecanol; and
higher fatty acids such as oleic acid, linoleic acid or linolenic acid, and mixtures thereof.

The non-volatile silicone oils that may be used in the composition according to the present disclosure may be non-volatile polydimethylsiloxanes (PDMS); polydimethylsiloxanes comprising alkyl or alkoxy groups, which are pendent and/or at the end of a silicone chain, these groups each comprising from 2 to 24 carbon atoms, phenyl silicones, for instance phenyl trimethicones, phenyl dimethicones, phenyltrimethylsiloxydiphenylsiloxanes, diphenyl dimethicones and diphenylmethyldiphenyltrisiloxanes, and mixtures thereof.

The at least one non-volatile oil may be present in the composition according to the present disclosure in a content ranging from 1% to 50% by weight, for example, ranging from 2% to 30% by weight and for instance, from 3% to 20% by weight relative to the total weight of the composition.

The at least one liquid fatty phase may be present in the composition according to the present disclosure in a total amount ranging from 10% to 95% by weight, for example, from 20% to 90% by weight and for instance, from 30% to 85% by weight relative to the total weight of the composition.

### Fatty Substances

The composition according to the present disclosure may also comprise fatty substances other than the oils mentioned above, for example, but not limited to, waxes or pasty fatty substances.

As used herein, the term “waxes” means a fatty substance that is solid at room temperature.

The pasty fatty substances may be defined as substances having at least one of the following physicochemical properties:
a viscosity of from 0.1 to 40 Pa·s (1 to 400 poises), measured at 40° C. with a Contraves TV rotary viscometer equipped with a MS-r3 or MS-r4 spindle at a frequency of 60 Hz,
a melting point of 25-70° C., for instance, 25-55° C.

Waxes that may be used according to the present disclosure, include, but are not limited to:
waxes of animal origin such as beeswax, spermaceti, lanolin wax and lanolin derivatives, plant waxes such as carnauba wax, candelilla wax, ouricury wax, Japan wax, cocoa butter, cork fiber wax or sugar cane wax;
mineral waxes, for example paraffin wax, petroleum jelly wax, lignite wax, microcrystalline waxes and ozokerites,
synthetic waxes, for example, polyethylene waxes, and waxes obtained by Fisher-Tropsch synthesis;
silicone waxes, for example, substituted linear polysiloxanes; non-limiting examples that may be mentioned include polyether silicone waxes, alkyl or alkoxy dimethicones comprising from 16 to 45 carbon atoms, and alkyl methicones, for instance the C₃₀-C₄₅ alkyl methicone sold under the trade name AMS C30 by Dow Corning,
hydrogenated oils that are solid at 25° C., such as hydrogenated castor oil, hydrogenated jojoba oil, hydrogenated palm oil, hydrogenated tallow, hydrogenated coconut oil fatty esters that are solid at 25° C., for instance the C₂₀-C₄₀ alkyl stearate sold under the trade name Kester Wax K82H by the company Koster Keunen,
and/or mixtures thereof.

In at least one embodiment, polyethylene waxes, microcrystalline waxes, carnauba waxes, hydrogenated jojoba oil, candelilla waxes and beeswaxes, and/or mixtures thereof, may be used.

Without wishing to be limited by any theory, the at least one wax may make it possible to reinforce the cicatrization properties and to reduce the tacky to pasty nature of the compositions according to the present disclosure.

In at least one embodiment, the at least one wax is present in a total amount ranging from 0.1% to 30% by weight, for example, from 0.5% to 20% by weight relative to the total weight of the composition.

The at least one fatty substances may be chosen in a varied manner by a person skilled in the art in order to prepare a composition having the desired properties, for example in terms of consistency or texture.

### Additional Thickener

In addition to the at least one bis-urea compound described above, the composition according to the present disclosure may comprise at least one additional oil thickener chosen from polymeric thickeners and mineral thickeners.

The polymeric oil thickener is capable of thickening or gelling the organic phase of the composition. The polymeric thickener may also be film-forming, i.e. it may be capable of forming a film during its application to the skin.

The at least one polymeric oil thickener may be chosen from, for example:
polycondensates of polyamide type resulting from condensation between (a) at least one acid chosen from dicarboxylic acids comprising at least 32 carbon atoms, such as fatty acid dimers, and (b) an alkylenediamine such as ethylenediamine, in which the polyamide polymer comprises at least one carboxylic acid end group esterified or amidated with at least one saturated and linear monoalcohol or one saturated and linear monoamine comprising from 12 to 30 carbon atoms, for example, ethylenediamine/stearyl dilinoleate copolymers such as the product sold under the name Uniclear 100 VG® by the company Arizona Chemical;
silicone polymers such as:
   1) polyorganosiloxanes comprising at least two groups capable of establishing hydrogen interactions, these two groups being located in the polymer chain, and/or
   2) polyorganosiloxanes comprising at least two groups capable of establishing hydrogen interactions, these two groups being located on grafts or branches on the polymer.

Groups capable of establishing hydrogen interactions may be chosen from ester, amide, sulfonamide, carbamate, thiocarbamate, urea, urethane, thiourea, oxamido, guanidino and biguanidino groups, and combinations thereof.

By way of non-limitiing example, the silicone polymers which may be used as structuring agents in the composition of the present disclosure are polymers of the polyorganosiloxane type, for instance those described in U.S. Pat. No. 5,874,069, U.S. Pat. No. 5,919,441, U.S. Pat. No. 6,051,216 and U.S. Pat. No. 5,981,680.

For example, in one embodiment, the silicone polymers are polyorganosiloxanes as defined above in which the units capable of establishing hydrogen interactions are located in the polymer chain.

According to one embodiment of the present disclosure, the silicone polymers may be polymers comprising at least one unit corresponding to the formula VI:

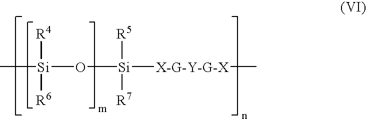

wherein:
1) R⁴, R⁵, R⁶ and R⁷, which may be identical or different, are chosen from:
   linear, branched and cyclic, saturated or unsaturated, C₁ to C₄₀ hydrocarbon-based groups, optionally comprising in their chain at lest one entity chosen from oxygen, sulfur and/or nitrogen atoms, and optionally substituted with fluorine atoms,
   C₆ to C₁₀ aryl groups, optionally substituted with at least one group chosen from C₁ to C₄ alkyl groups,
   polyorganosiloxane chains optionally comprising at least one entity chosen from oxygen, sulfur and/or nitrogen atoms;
2) the groups X, which may be identical or different, are chosen from linear and branched C₁ to C₃₀ alkylenediyl group, each optionally comprising in their respective chains at least one heteroatom chosen from oxygen and/or nitrogen atoms;
3) Y is chosen saturated or unsaturated, C₁ to C₅₀ linear and branched divalent alkylene, arylene, cycloalkylene, alkylarylene or arylalkylene group, optionally comprising at least one heteroatom chosen from oxygen, sulfur and/or nitrogen atoms, and optionally bearing at least one substituent chosen from one of the following: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl, C₁ to C₃ hydroxyalkyl and C₁ to C₆ aminoalkyl groups; or
4) Y represents a group of formula:

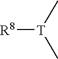

wherein:
T is chosen from linear and branched, saturated or unsaturated, C₃ to C₂₄ trivalent or tetravalent hydrocarbon-based group optionally substituted with a polyorganosiloxane chain, and optionally comprising at least one atom chosen from O, N and S, or, alternatively, T is a trivalent atom chosen from N, P and Al; and
R⁸ is chosen from linear and branched C₁ to C₅₀ alkyl group and polyorganosiloxane chains, optionally comprising at least one group chosen from: ester, amide, urethane, thiocarbamate, urea, thiourea and/or sulfonamide groups, which may optionally be linked to another chain of the polymer;
5) the groups G, which may be identical or different, are divalent groups chosen from:

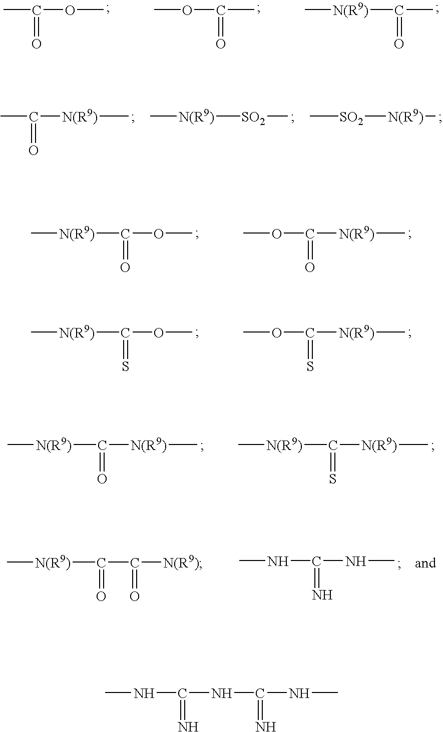

wherein R⁹ is chosen from hydrogen and linear and branched C₁ to C₂₀ alkyl groups, wherein at least 50% of the groups R⁹ of the polymer represent a hydrogen atom and at least two of the groups G of the polymer are a group other than:

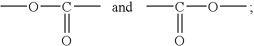

6) n is an integer ranging from 2 to 500, for example from 2 to 200, and m is an integer ranging from 1 to 1000, for example from 1 to 700, for instance, from 6 to 200.

According to one embodiment of the present disclosure, 80% of the groups R⁴, R⁵, R⁶ and R⁷ of the polymer are chosen from methyl, ethyl, phenyl and 3,3,3-trifluoropropyl groups.

In one embodiment, the groups capable of establishing hydrogen interactions are amide groups of formulae —C(O)NH— and —HN—C(O)—.

In this case, the at least one structuring agent may be chosen from polymers comprising at least one unit chosen from formula (VII) or (VIII):

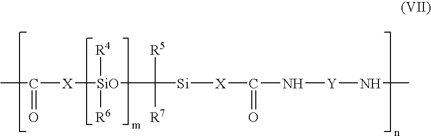

or

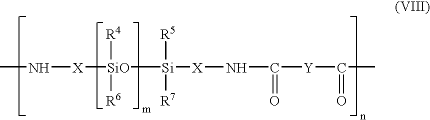

wherein R⁴, R⁵, R⁶, R⁷, X, Y, m and n are defined as in formula (VI).

In these polyamides of formula (VII) or (VIII), m ranges from 1 to 700, for instance, from 15 to 500 and for example from 50 to 200, and n ranges from 1 to 500, for example, from 1 to 100 and for instance from 4 to 25,
X, for example, may be chosen from linear and branched alkylene chains comprising from 1 to 30 carbon atoms, in instance 1 to 20 carbon atoms, for example from 5 to 15 carbon atoms such as 10 carbon atoms, and
Y, for example, may be chosen from linear and branched alkylene chains, optionally comprising rings and/or unsaturations, comprising from 1 to 40 carbon atoms, for example from 1 to 20 carbon atoms, for instance, from 2 to 6 carbon atoms, such as 6 carbon atoms.

According to another embodiment, the structuring agent may be chosen from galactomannans comprising from one to six, for example, from two to four hydroxyl groups per saccharide, substituted with a saturated or unsaturated alkyl chain, for instance guar gum alkylated with C₁-C₆ and in particular C₁-C₃ alkyl chains, and mixtures thereof.

The composition according to the present disclosure may also comprise at least one mineral oil thickener such as an organophilic clay or fumed silicas.

Organophilic clays, as disclosed herein, are clays modified with chemical compounds that make the clay able to swell in oily media.

Clays are products that are already well known per se, and are described, for example, in the publication “Minéralogie des argiles [Mineralogy of clays], S. Caillère, S. Hénin, M. Rautureau, 2nd Edition 1982, Masson”, the teachings of which are included herein by way of reference.

Clays are silicates comprising a cation that may be chosen from calcium, magnesium, aluminium, sodium, potassium and lithium cations, and mixtures thereof.

Non-limiting examples of clays that may be used includes clays of the smectite family such as montmorillonites, hectorites, bentonites, beidellites and saponites, and also of the vermiculite, stevensite and chlorite families.

The clays may be of natural or synthetic origin. Clays as used in the compositions of the present disclosure are cosmetically compatible and acceptable with keratin materials such as the skin.

The at least one organophilic clay may be chosen from montmorillonite, bentonite, hectorite, attapulgite and sepiolite. In one embodiment of the present disclosure, the clay is chosen from bentonite and hectorite.

The organophilic clays may be modified with a chemical compound chosen from quaternary amines, tertiary amines, amine acetates, imidazolines, amine soaps, fatty sulfates, alkyl aryl sulfonates and amine oxides, and mixtures thereof.

Organophilic clays that may be used in compositions according to the present disclosure include, but not limited to: quaternium-18 bentonites such as those sold under the names Bentone 3, Bentone 38 and Bentone 38V , by the company Rheox, Bentone ISD V by the company Elementis, Tixogel VP by the company United Catalyst, and Claytone 34, Claytone 40 and Claytone XL by the company Southern Clay; stearalkonium bentonites such as those sold under the names Bentone 27 by the company Rheox, Tixogel LG by the company United Catalyst and Claytone AF and Claytone APA by the company Southern Clay; quaternium-18/benzalkonium bentonites such as those sold under the names Claytone HT and Claytone PS by the company Southern Clay.

The fumed silicas may be obtained by high-temperature hydrolysis of a volatile silicon compound in an oxhydric flame, producing a finely divided silica. This process makes it possible for example to obtain hydrophilic silicas having a large number of silanol groups at their surface. Such hydrophilic silicas are sold, for example, under the names Aerosil 130®, Aerosil 200®, Aerosil 255®, Aerosil 300® and Aerosil 380® by the company Degussa, and Cab-O-Sil HS-5®, Cab-O-Sil EH-5®, Cab-O-Sil LM-130®, Cab-O-Sil MS-55® and Cab-O-Sil M-5® by the company Cabot.

The surface of the said silicas may be chemically modified via a chemical reaction generating a reduction in the number of silanol groups. For example, it is possible to substitute silanol groups with hydrophobic groups to obtain a hydrophobic silica.

Non-limiting mention may be made of hydrophobic groups including:
trimethylsiloxyl groups, which are obtained for example, by treating fumed silica in the presence of hexamethyldisilazane. Silicas thus treated are known as “silica silylate” according to the CTFA (6th Edition, 1995). They are sold, for example, under the name Aerosil R812® by the company Degussa and Cab-O-Sil TS-530® by the company Cabot;
dimethylsilyloxyl or polydimethylsiloxane groups, which are obtained, for example, by treating fumed silica in the presence of polydimethylsiloxane or dimethyldichlorosilane. Silicas thus treated are known as “silica dimethyl silylate” according to the CTFA (6th Edition, 1995). They are sold, for example, under the references Aerosil R972® and Aerosil R974® by the company Degussa and Cab-O-Sil TS-610® and Cab-O-Sil TS-720® by the company Cabot.

In at least one embodiment, the fumed silica have a particle size that may range from nanometric to micrometric, for example ranging from about 5 to 200 nm.

Without wishing to be limited by any theory, the mineral thickeners may make it possible to reinforce the cicatrization properties and to reduce the tacky to pasty nature of the compositions according to the present disclosure.

The at least additional oil thickener may be present in the composition according to the present disclosure in an active material content ranging from 0.01% to 15% by weight, for example, from 0.1% to 10% by weight and for instance, from 0.3% to 5% by weight relative to the total weight of the composition.

### Aqueous Phase

The composition according to the present disclosure may comprise at least one aqueous phase.

The at least one aqueous phase comprises water. For example, the water may be a floral water such as cornflower water and/or a mineral water such as eau de Vittel, eau de Lucas or eau de La Roche Posay and/or a spring water.

The at least one aqueous phase may also comprise at least one organic solvent that is water-miscible (at room temperature −25° C.), for instance monoalcohols comprising from 2 to 6 carbon atoms such as ethanol or isopropanol; polyols comprising, for example, from 2 to 20 carbon atoms, for instance, from 2 to 10 carbon atoms such as from 2 to 6 carbon atoms, such as glycerol, propylene glycol, butylene glycol, pentylene glycol, hexylene glycol, dipropylene glycol or diethylene glycol; and glycol ethers (for example, comprising from 3 to 16 carbon atoms), such as (C₁-C₄) alkyl ethers of monopropylene, dipropylene or tripropylene glycol, and (C₁-C₄)alkyl ethers of monoethylene, diethylene or triethylene glycol, and mixtures thereof.

The aqueous phase may also comprise at least one stabilizer, for example sodium chloride, magnesium dichloride or magnesium sulfate.

The aqueous phase may also comprise any water-soluble or water-dispersible compound that is compatible with an aqueous phase, such as gelling agents, film-forming agents, thickeners or surfactants, and mixtures thereof.

The at least one aqueous phase may be present in the composition, according to on embodiment of the present disclosure, in an amount ranging from 1% to 80% by weight, for example, from 5% to 50% by weight, such as 5% to 40% by weight relative to the total weight of the composition.

In another embodiment, the composition according to the present disclosure may also be anhydrous. As disclosed herein, the term “anhydrous composition” means a composition comprising less than 5% by weight of water, for example, less than 2% by weight of water relative to the total weight of the composition. In still another embodiment, the composition is free of water, the water not being added during the preparation of the composition, but corresponding to the residual water provided by the mixed ingredients.

According to at least one embodiment, the at least one aqueous phase and the at least one oily phase may be present in an amount such that the weight ratio of water to oil is greater than or equal to 1 and for example between 1 and 1.2.

### Pulverulent Phase

The composition according to the present disclosure may comprise a least one pulverulent phase comprising, for example, pigments, fillers and nacres, and mixtures thereof.

According to at least one embodiment, the composition according to the present disclosure comprises at least one pigment.

As used herein, the term “pigments” means mineral or organic particles, which are insoluble in the liquid organic phase, and which may color and/or opacify the composition.

The pigments may be mineral or organic pigments. Pigments that may be used in the composition disclosed herein include metal oxides, for instance iron oxides (for example, yellow, red, brown and black iron oxides), titanium dioxides, cerium oxide, zirconium oxide and chromium oxide; manganese violet, ultramarine blue, Prussian blue, cobalt blue and ferric blue, and mixtures thereof.

In at least one embodiment, the at least one pigment is chosen from iron oxides and titanium oxide pigments.

The pigments may be treated with a hydrophobic agent to make them compatible with the organic phase of the composition. The hydrophobic-treatment agent may be chosen from silicones, for instance methicones, dimethicones and perfluoroalkylsilanes; fatty acids, for instance stearic acid; metal soaps, for instance aluminium dimyristate, and the aluminium salt of hydrogenated tallow glutamate, perfluoroalkyl phosphates, perfluoroalkylsilanes, perfluoroalkylsilazanes, polyhexafluoropropylene oxides, polyorganosiloxanes comprising perfluoroalkyl perfluoropolyether groups, and amino acids; N-acylamino acids or salts thereof; lecithin, isopropyl triisostearyl titanate, and mixtures thereof.

The N-acylamino acids used in the compositions disclosed herein may comprise an acyl group comprising from 8 to 22 carbon atoms, for instance a 2-ethylhexanoyl, caproyl, lauroyl, myristoyl, palmitoyl, stearoyl or cocoyl group. The salts of these compounds may be aluminium, magnesium, calcium, zirconium, zinc, sodium or potassium salts. The amino acid may be, for example, lysine, glutamic acid or alanine.

In at least one embodiment, the term “alkyl” mentioned in the compounds mentioned above for example denotes an alkyl group comprising from 1 to 30 carbon atoms and for example, comprising from 5 to 16 carbon atoms.

Hydrophobic-treated pigments used in the compositions disclosed herein are described for example in the European Patent Application EP-A-1,086,683.

The at least one pigment in the composition according to the present disclosure may be present in a amount ranging from 0.1% to 40% by weight, for example, from 1% to 30% by weight and for instance from 5% to 15% by weight relative to the total weight of the composition.

In addition to pigments, the at least one pulverulent phase of the composition according to the present disclosure may comprise fillers and/or nacres.

According to at least one embodiment, the composition according to the present disclosure may comprise at least one filler.

The term “fillers” should be understood as meaning colorless or white, mineral or synthetic particles of any form, which are insoluble in the medium of the composition irrespective of the temperature at which the composition is manufactured.

The fillers may be mineral or organic and of any form, platelet-shaped, spherical or oblong, irrespective of the crystallographic form (for example lamellar, cubic, hexagonal, orthorhombic, etc.). Illustrative examples of fillers suitable for the composition disclosed herein include, but are not limited to: talc, mica, silica, kaolin, polyamide (Nylon®) powders, poly-β-alanine powders, polyethylene powders, polymethyl methacrylates, polyurethane powders such as the powder of the copolymer of hexamethylene diisocyanate and of trimethylol hexyl lactone sold under the name Plastic Powder D-400 by the company Toshiki, tetrafluoroethylene polymer (Teflon®) powders, micronized wax particles, for example carnauba microwaxes such as those sold under the name MicroCare 350® by the company Micro Powders, microwaxes of synthetic wax such as those sold under the name MicroEase 114S® by the company Micro Powders, microwaxes consisting of a mixture of carnauba wax and of polyethylene wax, such as those sold under the names MicroCare 300® and 310® by the company Micro Powders, microwaxes consisting of a mixture of carnauba wax and of synthetic wax, such as those sold under the name MicroCare 325® by the company Micro Powders, polyethylene microwaxes such as those sold under the names MicroPoly 200®, 220®, 220L® and 250S® by the company Micro Powders, and those sold under the name Cerapure H5-C by the company Shamrock, and polypropylene microwaxes such as those sold under the name Mattewax by the company Micro Powders; lauroyllysine, starch, boron nitride, hollow polymer microspheres such as those of polyvinylidene chloride/acrylonitrile, for instance Expancel® (Nobel Industrie), powders of acrylic acid copolymers, silicon resin powders, in particular silsesquioxane powders (silicon resin powders described for example, in patent EP 293795; for example Tospearls® from Toshiba), elastomeric polyorganosiloxane particles, precipitated calcium carbonate, magnesium carbonate, magnesium hydrogen carbonate, hydroxyapatite, hollow silica microspheres, glass or ceramic microcapsules, and metal soaps derived from organic carboxylic acids comprising from 8 to 22 carbon atoms and for example, from 12 to 18 carbon atoms, for example zinc stearate, magnesium stearate, lithium stearate, zinc laurate or magnesium myristate; barium sulfate, and mixtures thereof.

In at least one embodiment, the composition according to the present disclosure may comprise a polytetrafluoroethylene (PTFE) powder.

The at least one filler may be present in the composition according to the present disclosure in a total content ranging from 0.1% to 30% by weight, for example, from 0.5% to 20% by weight and for instance, from 0.8% to 10% by weight relative to the total weight of the composition.

In addition to the pigments and fillers, the at least one particulate phase of the composition according to the present disclosure may comprise at least one nacres.

As used herein, the term “nacres” means iridescent particles, for example produced by certain molluscs in their shell or else synthesized, which are insoluble in the medium of the composition.

The nacres may be chosen from white nacreous pigments such as bismuth oxychloride, mica coated with titanium or with bismuth oxychloride, colored nacreous pigments such as titanium mica with iron oxides, titanium mica for example, with ferric blue or with chromium oxide or titanium mica with an organic pigment of the abovementioned type, and also nacreous pigments based on bismuth oxychloride.

### Dyes

The composition according to the present disclosure may further comprise at least one dyes chosen from water-soluble and liposoluble dyes.

The water-soluble dyes maybe chosen from, for example, beetroot juice, methylene blue and caramel.

As used herein, the term “liposoluble dyes” should be understood as meaning generally organic compounds that are soluble in fatty substances such as oils.

The liposoluble dyes maybe chosen from, for example, Sudan red, D&C Red No 17, D&C Green No 6, β-carotene, soybean oil, Sudan brown, D&C Yellow No 11, D&C Violet No 2, D&C Orange No 5, quinoline yellow, annatto and bromo acids.

### Galenicals Forms

The composition according to the present disclosure may be in various galenical forms, for example, anhydrous or, for example, in the form of water-in-oil emulsions, or multiple emulsions with an oily continuous phase, and may be in the form of gels, creams or suspensions, in compact or hot-cast form or in the form of sticks.

The composition according to the present disclosure may be in the form of a makeup composition, for example a complexion product such as a foundation, a makeup rouge or an eyeshadow; a lip product such as a lipstick, a lip gloss or a lipcare product; a concealer product; a blusher, a mascara or an eyeliner; an eyebrow makeup product, a lip pencil or an eye pencil; a nail product such as a nail varnish or a nailcare product; a body or hair makeup product (hair lacquer or mascara); a skincare protecting composition for the face, the neck, the hands or the body, for example, an anti-wrinkle, anti-fatigue or anti-ageing composition for making the skin look radiant, or a moisturizing or medicated composition; an antisun, after-sun or artificial tanning composition; a hair composition, for example for hair dyeing, haircare or hair hygiene, for styling or holding the hairstyle or for shaping the hair.

### Additional Cosmetic Adjuvants

The composition according to the present disclosure may comprise at least one cosmetic adjuvant, which may be chosen for example from antioxidants, fragrances, preserving agents, neutralizers, surfactants, sunscreens, vitamins, moisturizers, self-tanning compounds, anti-wrinkle active agents, emollients, hydrophilic or lipophilic active agents, free-radical scavengers, deodorants, sequestrants and film-forming agents, and mixtures thereof.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to limit the application of the doctrine of equivalents to the scope of the claims, each numericval parameter should be construed in light of the number of significant digits and ordinary rounding approaches.

Notwithstanding that the numerical ranges and parameters setting forth the broad scope of the present disclosure are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLES

Unless indicated otherwise, the amounts indicated are expresses as percentages by mass.

### EXAMPLE 1

A foundation having the composition below was prepared:

| | | Concentration |
|---|---|---|
| PHASE | Name | (mass %) |
| A1 | Compound of formula (I) in the form of a mixture | 1.50 |
| | predominantly constituted of the compounds: | |
| | 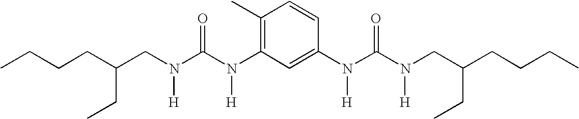 | |
| | 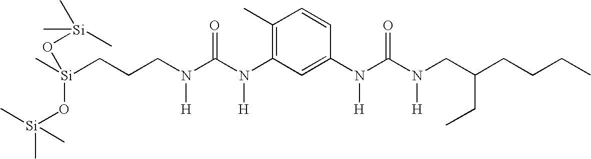 | |
| | and | |
| | 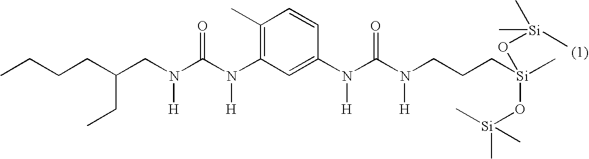 | |
| | Parleam oil | 32.10 |
| A2 | Cetyl dimethicone copolyol (2) | 1.80 |
| | Mono/diglycerides of isostearic | 0.60 |
| | acid esterified with succinic acid (3) | |
| A3 | Cyclopentasiloxane | 5.00 |
| | Iron oxide pigments | 2.04 |
| | Titanium dioxide pigments | 7.96 |
| A4 | Nylon 12 | 8.00 |
| B | Water | 40.00 |
| | Preserving agents | 1.00 |
| | TOTAL | 100.000 |

(1) The preparation of mixture (l) is descibed in the procedure below.
(2) Sold under the name Abil EM 90 by the company Goldschmidt.
(3) Sold under the name Imwitor 780K by the company Sasol.

### Procedure:

### 1. Preparation of the Compound of General Formula (I)

The preparation of compounds of bis-urea type, comprising at least one silicone unit and at least one non-silicone unit, was performed.

The mixture is obtained in a single step, the synthetic scheme being as follows:

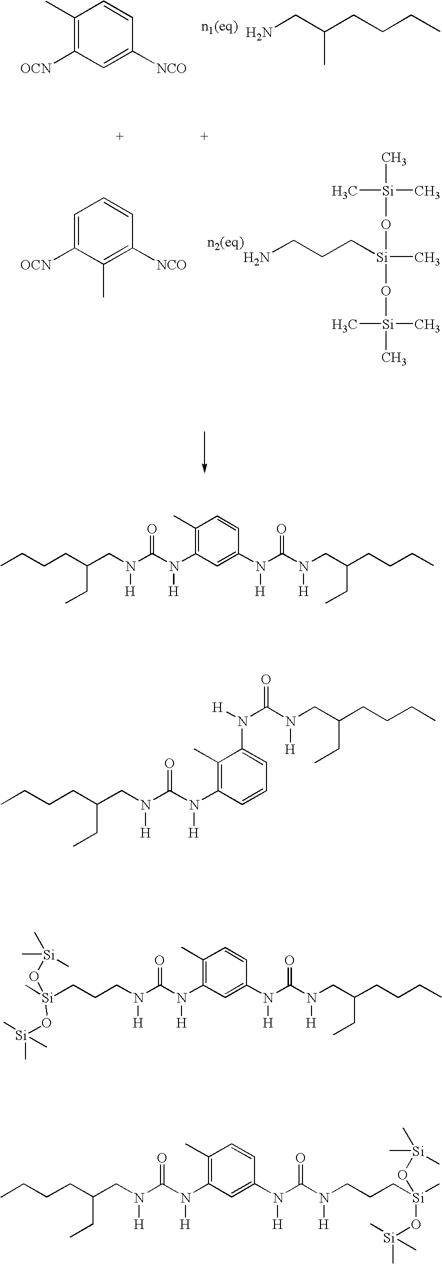

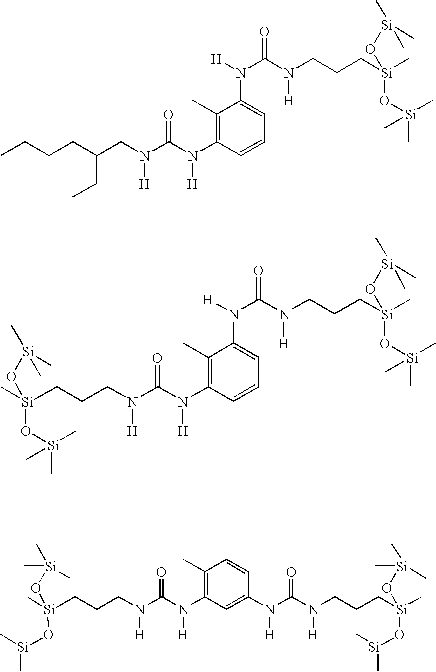

The starting materials are as follows:
tolylene diisocyanate (TDI): 95/5 (2,4 isomer)/(2,6 isomer); m=50 g (0.29 mol);
n₁=0.44 mol (1.5 eq.) i.e. m=56.87 g of 2-ethyl-hexylamine;
n₂=0.19 mol (0.66 eq.) i.e. m=53.13 g of 3-amino-propylmethylbis(trimethylsiloxy)silane with a content of β isomer of less than 1%.

This thus gives n_{R}/n′_{R}=2.23 (69/31).

The tolylene diisocyanate dissolved in anhydrous THF was mixed with 2.2 equivalents of amine dissolved in anhydrous THF.

The reaction is performed under an inert atmosphere (argon) in anhydrous medium with a reaction medium temperature maintained between 15° C. and 40° C.

In parallel, a solution of amine (Y) in THF was prepared. Since the temperature of the reaction medium should for example, not exceed 40° C., the concentration of the amine and the rate of addition of the amine solution (Y) are adjusted to this requirement. The reaction medium was left stirring, while the reaction progress was monitored by infrared spectrometry (disappearance of the NCO band between 2250 and 2280 cm⁻¹).

Once the diisocyanate has completely reacted, the reaction mixture was added to water acidified (to pH 3) with hydrochloric acid, and the precipitate obtained was filtered off, washed several times with water and finally dried under vacuum or freeze-dried. A white powder is obtained and is used without further purification after analysis (HPLC coupled to mass spectrometry).

The mixture finally obtained comprises the following bis-ureas:

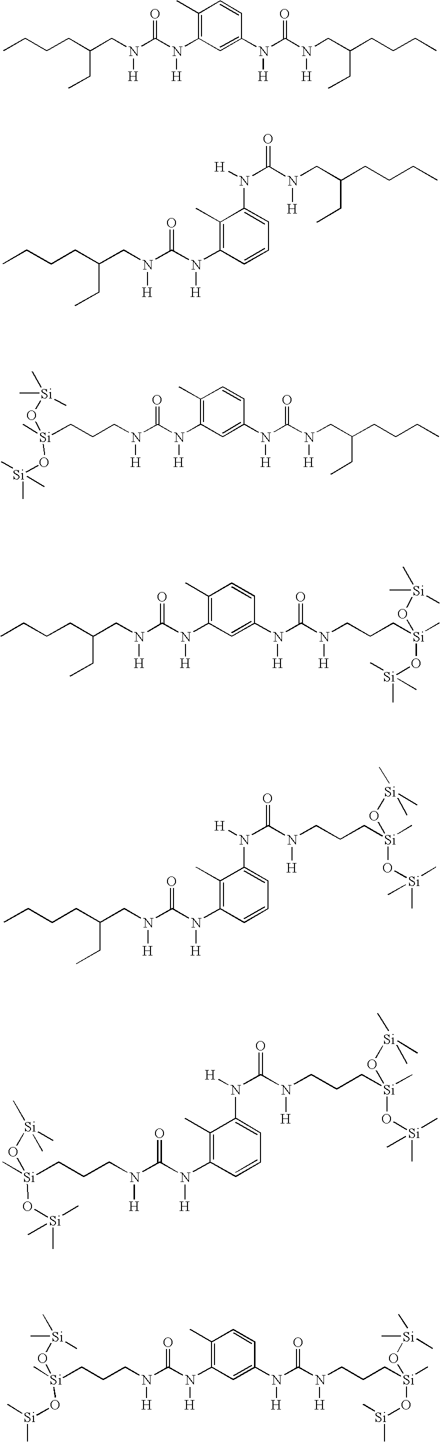

### 2. Preparation of the Foundation Composition

The compounds of phase A1 are melted in a beaker, at a temperature of 100° C. with magnetic stirring for about one hour (phase A1).

The compounds of phase A2 are added to phase A1 with stirring using a Moritz blender, at a temperature of 80° C. (phases A1+A2).

The pigments of phase A3 are ground in cyclopentasiloxane in a three-roll mill, and phase A3 is then added to the mixture A1+A2.

The Nylon powder of phase A4 is added with continued stirring, by sprinkling it onto the mixture A1+A2+A3.

The water and the preserving agents are weighed out in a separate beaker. This beaker is placed on a hotplate with magnetic stirring in order to dissolve the preserving agents in the water. The mixture B is allowed to cool to room temperature and was then poured slowly into the mixture A1+A2+A3+A4 to form the emulsion.

The foundation obtained has a creamy texture, which, when applied to the skin, forms a smooth film. In addition, after taking up on a finger the product in the jar (or manual shear) the surface of the product in the jar regains its initial shape (surface repair).

### EXAMPLE 2

A foundation having the composition below was prepared:

| | | Concentration |
|---|---|---|
| PHASE | Name | (mass %) |
| A1 | Compound of formula (I) in the form of a mixture | 2.50 |
| | predominantly constituted of the compounds: | |
| | 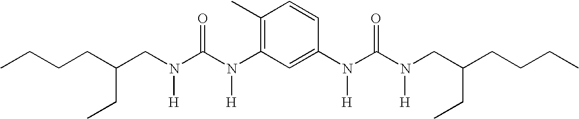 | |
| | 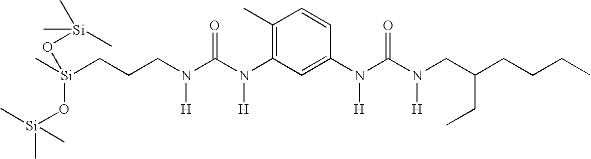 | |
| | and | |
| | 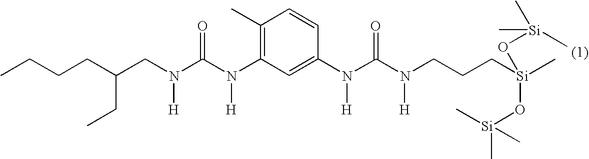 | |
| | Parleam oil | 31.10 |
| A2 | Cetyl dimethicone copolyol (2) | 1.80 |
| | Mono/diglycerides of isostearic | 0.60 |
| | acid esterified with succinic acid (3) | |
| A3 | Cyclopentasiloxane | 5.00 |
| | Iron oxide pigments | 2.04 |
| | Titanium dioxide pigments | 7.96 |
| A4 | Nylon 12 | 8.00 |
| B | Water | 40.00 |
| | Preserving agents | 1.00 |
| | TOTAL | 100.000 |

(1) The preparation of mixture (l) was performed according to the same protocol as Example 1.
(2) Sold under the name Abil EM 90 by the company Goldschmidt.
(3) Sold under the name Imwitor 780K by the company Sasol.

The procedure for obtaining the foundation is also the same as that of Example 1.

The foundation obtained has a creamy texture, which, when applied to the skin, forms a smooth film. In addition, after taking up on a finger the product in the jar (manual shear), the surface of the product in the jar regains its initial shape (surface repair).

### EXAMPLE 3

A foundation having the composition below was prepared:

| | | Concentration |
|---|---|---|
| PHASE | Name | (mass %) |
| A1 | Compound of formula (I) in the form of a mixture | 1.50 |
| | predominantly constituted of the compounds: | |
| | 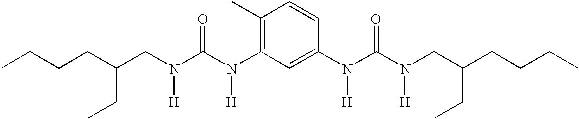 | |
| | 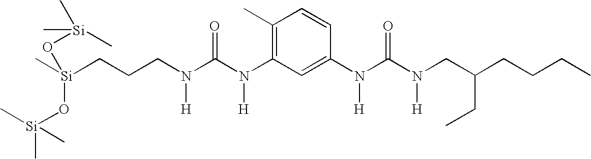 | |
| | and | |
| | 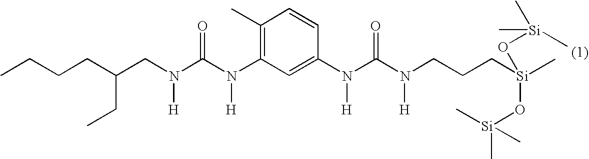 | |
| | Cyclopentasiloxane | 32.10 |
| A2 | Cetyl dimethicone copolyol (2) | 1.80 |
| | Mono/diglycerides of isostearic | 0.60 |
| | acid esterified with succinic acid (3) | |
| A3 | Cyclopentasiloxane | 5.00 |
| | Iron oxide pigments | 2.04 |
| | Titanium dioxide pigments | 7.96 |
| A4 | Nylon 12 | 8.00 |
| B | Water | 40.00 |
| | Preserving agents | 1.00 |
| | TOTAL | 100.000 |

(1) The preparation of mixture (l) was performed according to the same protocol as Example 1.
(2) Sold under the name Abil EM 90 by the company Goldschmidt.
(3) Sold under the name Imwitor 780K by the company Sasol

The procedure for obtaining the foundation is also the same as that of Example 1.

The foundation obtained has a creamy texture, which, when applied to the skin, forms a smooth film. In addition, after taking up on a finger the product in the jar (manual shear), the surface of the product in the jar regains its initial shape (surface repair).

### EXAMPLE 4

A foundation having the composition below was prepared:

| | | Concentration |
|---|---|---|
| PHASE | Name | (mass %) |
| A1 | Compound of formula (I) in the form of a mixture | 2.50 |
| | predominantly constituted of the compounds: | |
| | 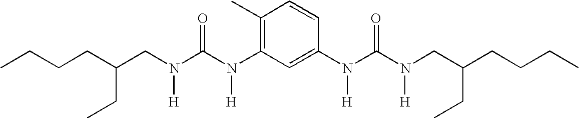 | |
| | 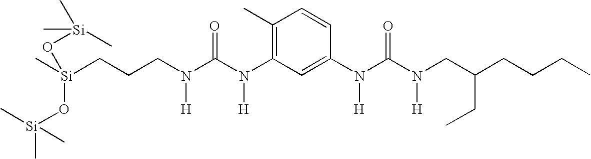 | |
| | and | |
| | 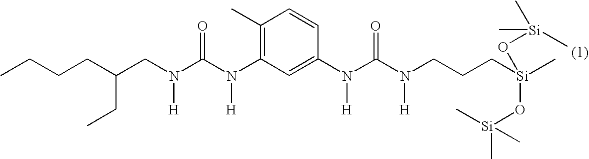 | |
| | Cyclopentasiloxane | 31.10 |
| A2 | Cetyl dimethicone copolyol (2) | 1.80 |
| | Mono/diglycerides of isostearic | 0.60 |
| | acid esterified with succinic acid (3) | |
| A3 | Cyclopentasiloxane | 5.00 |
| | Iron oxide pigments | 2.04 |
| | Titanium dioxide pigments | 7.96 |
| A4 | Nylon 12 | 8.00 |
| B | Water | 40.00 |
| | Preserving agents | 1.00 |
| | TOTAL | 100.000 |

(1) The preparation of mixture (l) was performed according to the same protocol as Example 1.
(2) Sold under the name Abil EM 90 by the company Goldschmidt.
(3) Sold under the name Imwitor 780K by the company Sasol.

The procedure for obtaining the foundation is also the same as that of Example 1.

The foundation obtained has a creamy texture, which, when applied to the skin, forms a smooth film. In addition, after taking up on a finger the product in the jar (manual shear), the surface of the product in the jar regains its initial shape (surface repair).

What is claimed is:

1. A cosmetic composition comprising, in a cosmetically acceptable medium, at least one continuous liquid fatty phase, comprising at least one compound of formula (I): 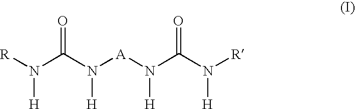
and salts and isomers thereof,
wherein:
A is chosen from groups of formula (II): 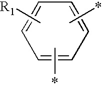
wherein R₁ is chosen from linear and branched C₁-C₄ alkyl radicals, and the asterisks, *, symbolize the points of attachment of the group A to each of the two nitrogen atoms of the rest of the compound of formula (I), and
R and R′, which may be identical or different, are chosen from:
i) the radicals of formula (III): 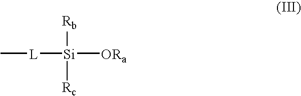
wherein:
L is chosen from a single bond, and linear, branched and cyclic, saturated and unsaturated, divalent hydrocarbon-based alkylene radicals, comprising 1 to 18 carbon atoms, and optionally comprising 1 to 4 heteroatoms chosen from N, O and S;
Rₐ is chosen from:
a) linear, branched and cyclic, saturated and unsaturated, hydrocarbon-based alkyl radicals, comprising 1 to 18 carbon atoms, optionally comprising 1 to 8 heteroatoms chosen from N, O, Si and S; and
b) silicone radicals of formula: 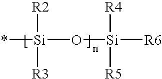
wherein n ranges from 0 to 100;
and wherein R₂ to R₆ are, independently of each other, chosen from linear and branched hydrocarbon-based alkyl radicals comprising 1 to 12 carbon atoms, and optionally comprising 1 to 4 heteroatoms;
R_{b} and R_{c} are, independently of each other, chosen from:
a) linear, branched and cyclic, saturated and unsaturated hydrocarbon-based alkyl radicals, comprising 1 to 18 carbon atoms, and optionally comprising 1 to 4 heteroatoms chosen from N, O, Si and S;
b) the radicals of formula: 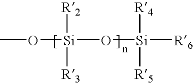
wherein n ranges from 0 to 100;
and R′₂ to R′₆ are, independently of each other, chosen from linear and branched hydrocarbon-based alkyl radicals, comprising 1 to 12 carbon atoms, and optionally comprising 1 to 4 heteroatoms; and
ii) linear, branched and cyclic, saturated and unsaturated, C₁-C₃₀ alkyl radicals, optionally comprising 1 to 3 heteroatoms chosen from O, S, F and N;
wherein at least one of the radicals R and R′ is chosen from those of formula (III); and at least one oil with a solubility parameter δₐ ranging from 0 to 7.00 (J/cm³)^{1/2}.

2. The composition according to claim 1, wherein the solubility parameter 67 ₐ of the at least one oil, ranges from 0 to 5.00 (J/cm³)^{1/2}.

3. The composition according to claim 1, wherein the group A is chosen from those of formulae 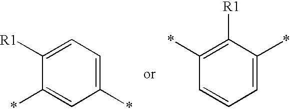
wherein R₁ is chosen from linear and branched C₁-C₄ alkyl radicals, and the asterisks, *, symbolize the points of attachment of the group A to each of the two nitrogen atoms of the rest of the compound of formula (I).

4. The composition according claim 1, wherein the group A is chosen from those of formulae:

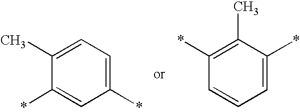

5. The composition according to claim 1, wherein L has the structure —(CH₂)n— with n=1 to 18; or alternatively L has the structure —CH₂-CH(CH₃)—.

6. The composition according to claim 1 wherein L is chosen from methylene, ethylene, propylene, butylene and octylene.

7. The composition according to claim 1, wherein Rₐ is chosen from radicals:
of structure —(CH₂)n′-CH₃ wherein n′=0 to 17;
of structures —(CH₂)x—O—(CH₂)z-CH₃ and —(CH₂)x—O'(CH₂)y—O—(CH₂)z-CH₃, wherein x=1 to 10; y=1 to 10, and z=0 to 10;
of structure -SiR₄R₅R₆, wherein R₄, R₅ and R₆ are, independently of each other, chosen from C₁-C₁₂ alkyl radicals; and
of formula: 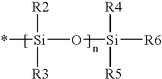
wherein R₂ to R₆ are, independently of each other, chosen from C₁-C₁₂ alkyl radicals.

8. The composition according to claim 1, wherein Rₐ is chosen from the structures: 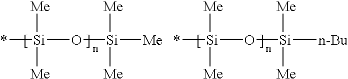
wherein n=1 to 100.

9. The composition according to claim 7, wherein Rₐ has the following structure:

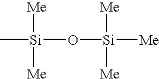

10. The composition according to claim 1, wherein R_{b} and R_{c}, which may be identical or different, are chosen from radicals:
of structure —(CH₂)m-CH₃ with m=0 to 17;
of structure —O—(CH₂)m′-CH₃ with m′=0 to 5;
of structures —O—(CH₂)x—O—(CH₂)z-CH₃ and —O—(CH₂)x—O—(CH₂)y—O—(CH₂)z-CH₃, wherein x =1 to 10; y=1 to 10, and z=0 to 10; and
of structure: 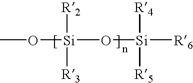
wherein n=0 to 100
and R′₂ to R′₆ are, independently of each other, chosen from C₁-C₁₂ alkyl radicals.

11. The composition according to claim 1, wherein at least one of the radicals R and R′ is chosen from the following radicals: 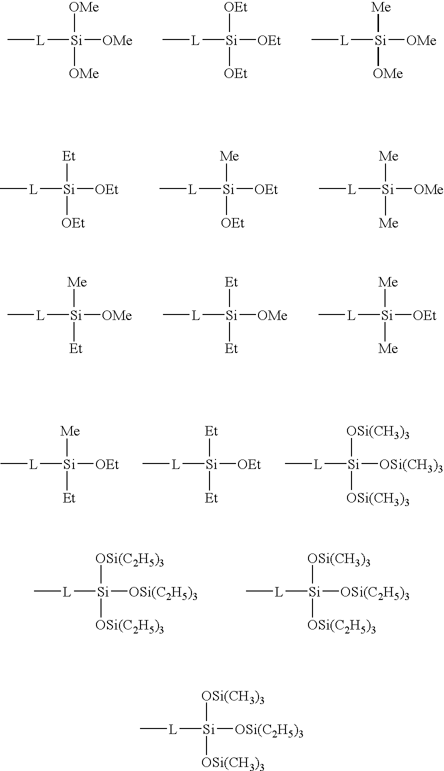
those of formulae: 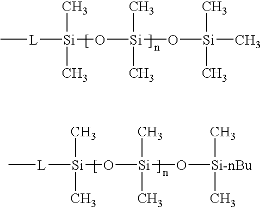
wherein n=0 to 100;
those of formulae: 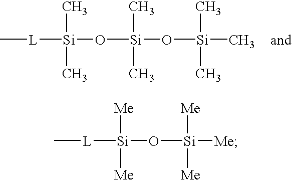
of formula: 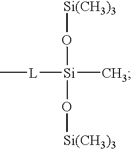
and those of formulae 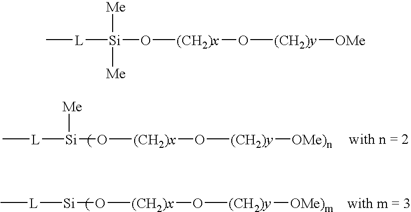
wherein x=1 to 10; and y=1 to 10;
and L is chosen from a single bond, and linear, branched and cyclic, saturated and unsaturated, divalent hydrocarbon-based alkylene radicals, comprising 1 to 18 carbon atoms, and optionally comprising 1 to 4 heteroatoms chosen from N, O and S.

12. The composition according to claim 11, wherein L is chosen from linear and branched C₁-C₈ alkylene radicals chosen from methylene, ethylene, propylene, butylene, and —CH₂-CH(CH₃)—.

13. The composition according to claim 1, wherein R and R′, which may be identical or different, are both of formula (III).

14. The composition according to claim 1, wherein at least one of the radicals R and R′ is chosen from linear, branched and cyclic, saturated and unsaturated, C₁-C₃₀ alkyl radicals, optionally comprising 1 to 3 heteroatoms chosen from O, S, F and N.

15. The composition according to claim 14, wherein the radicals R and R′ are chosen from: 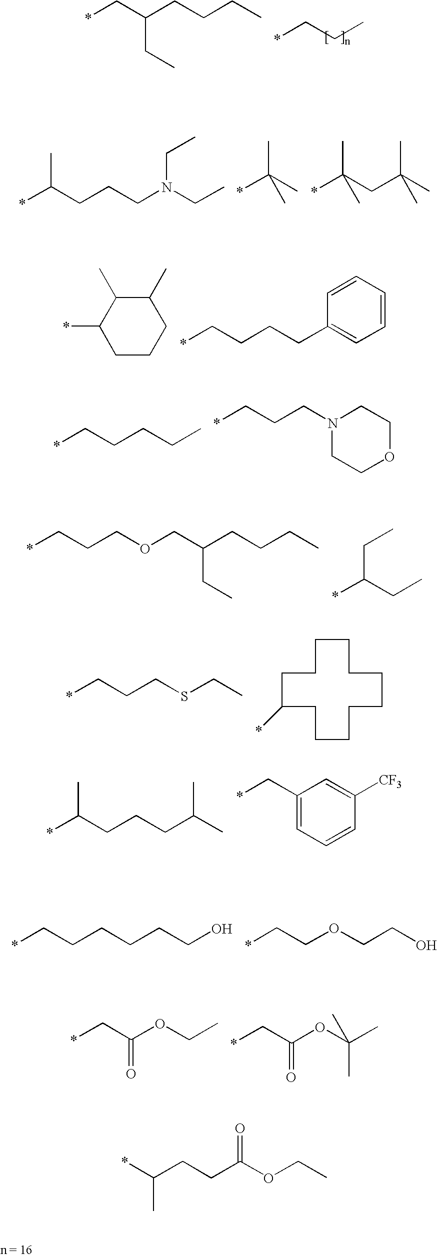
wherein the asterisk, *, symbolizes the point of attachment of each group to the nitrogen atoms of the rest of the compound of formula (I).

16. The composition according to claim 1, wherein the radicals R and R′ are chosen from branched, saturated and unsaturated alkyl radicals, comprising 3 to 16 carbon atoms, optionally comprising 1 to 3 heteroatoms chosen from O, S, F and N.

17. The composition according to claim 16, wherein the radicals R and R′ are chosen from tert-butyl radicals, 2-ethylhexyl radicals, and radicals of formula:

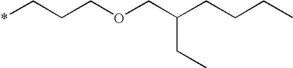

18. The composition according to claim 1, wherein the at least one compound of formula (I) is chosen from the following compounds, and salts and isomers thereof:

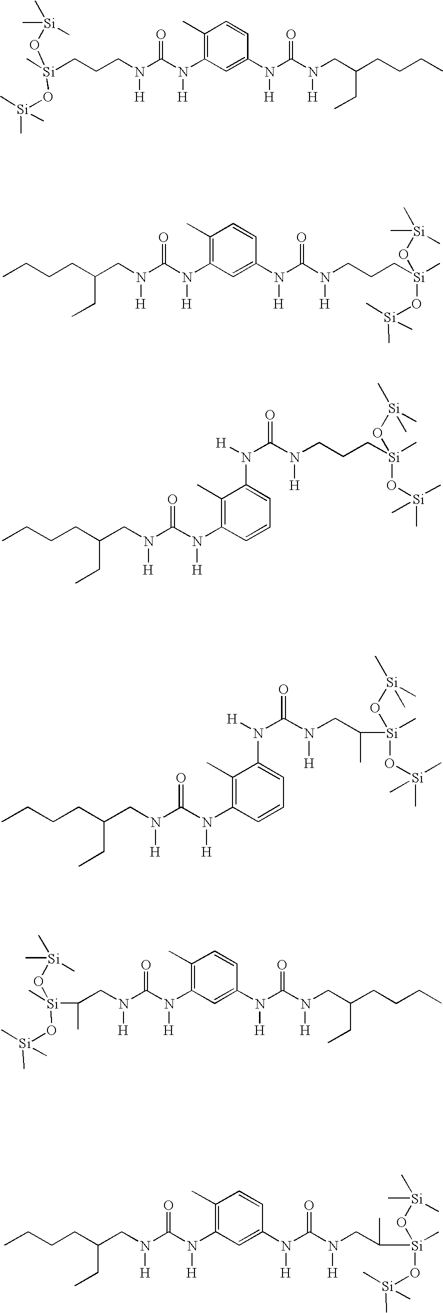

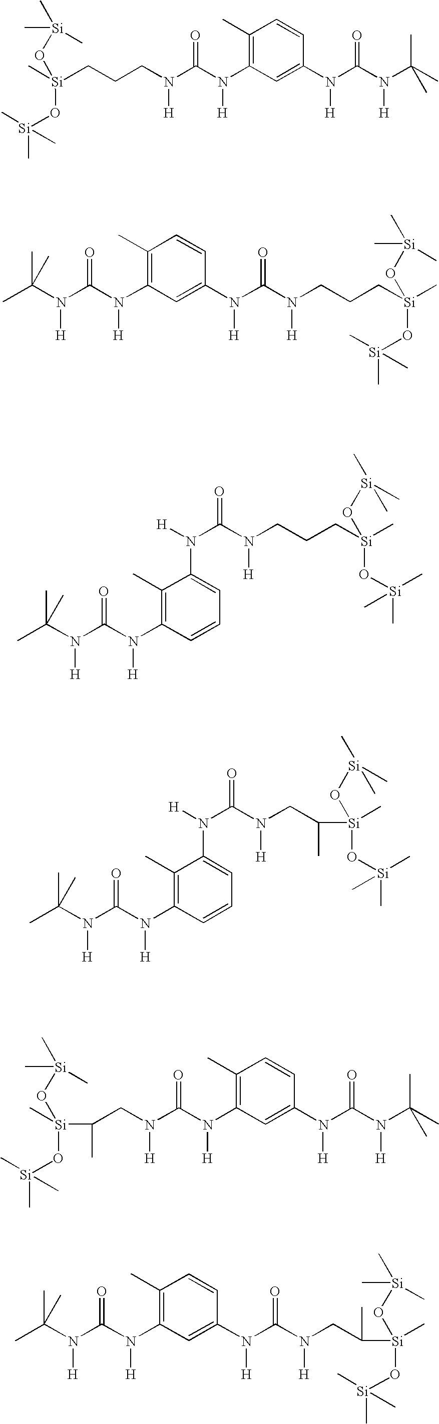

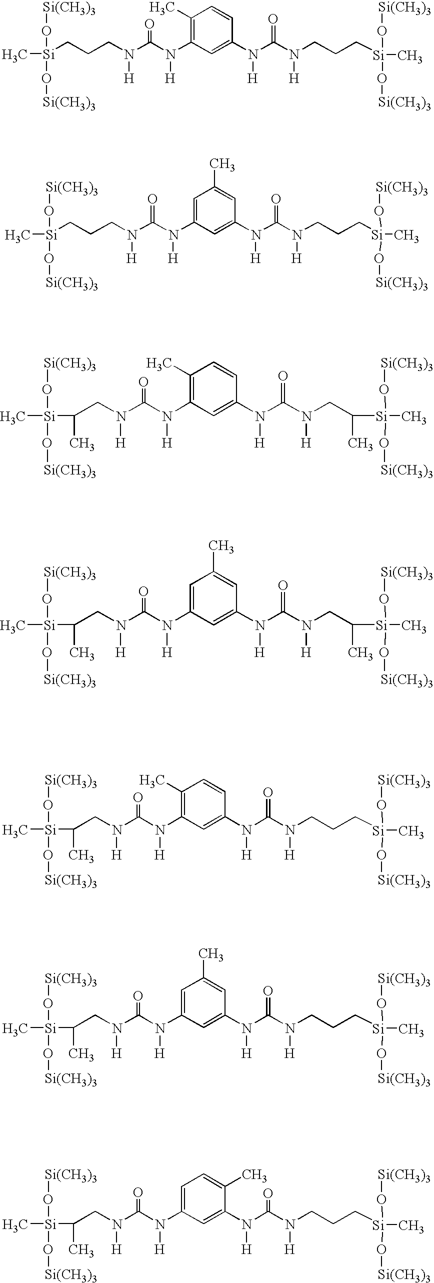

19. The composition according to claim 1, wherein the at least one compound of formula (I) is present in a total amount ranging from 0.01 % to 20% by weight relative to the total weight of the composition.

20. The composition according to claim 19, wherein the at least one compound of formula (i) is present in a total amount ranging from 2% to 5% by weight relative to the total weight of the composition.

21. The composition according to claim 1, wherein the at least one oil with a solubility parameter δₐ ranging from 0 to 7.00 (J/cm³)^{1/2} is chosen from hydrocarbon-based oils and silicone oils.

22. The composition according claim 1, wherein the at least one oil with a solubility parameter δₐ ranging from 0 to 7.00 (J/cm³)^{1/2} is chosen from aprotic alkanes.

23. The composition according to claim 22, wherein the at least one aprotic alkane has a molecular mass ranging from 165 to 900 g/mol.

24. The composition according to claim 22, wherein the at least one aprotic alkane is chosen from polyisobutylenes with a molecular weight ranging from 250 to 800 g/mol, squalane and parleam oil.

25. The composition according to claim 1, wherein the at least one oil with a solubility parameter δₐ ranging from 0 to 7.00 (J/cm³)^{1/2} is chosen from linear silicone oils.

26. The composition according to claim 25, wherein the at least one linear silicone oil is chosen from polydimethylsiloxanes, cyclopentadimethylsiloxane, cyclotetradimethylsiloxane, phenyl trimethicone and cyclohexadimethylsiloxane.

27. The composition according to claim 21, wherein the at least one silicone oil is branched with groups of low molecular weight.

28. The composition according to claim 25, wherein the at least one linear silicone oil with a solubility parameter δₐ ranging from 0 to 7.00 (J/cm³)^{1/2} has a molecular weight ranging from 100 to 800 g/mol.

29. The composition according to claim 1, wherein the at least one oil with a solubility parameter δₐ ranging from 0 to 7.00 (J/cm³)^{1/2} is present in the composition in an amount ranging from 20% to 100% by weight, relative to the total weight of the continuous liquid fatty phase.

30. The composition according to claim 29, wherein the at least one oil with a solubility parameter δₐ ranging from 0 to 7.00 (J/cm³)^{1/2} is present in the composition in an amount ranging from 60% to 95% by weight, relative to the total weight of the continuous liquid fatty phase.

31. The composition according to claim 1, further comprising at least one additional oil thickener different from the at least one compound of formula (I), chosen from polymeric thickeners and mineral thickeners.

32. The composition according to claim 31, wherein the at least one additional oil thickener is present in the composition in a total amount ranging from 0.1% to 10% by weight, relative to the total weight of the composition.

33. The composition according to claim 32, wherein the at least one additional oil thickener is present in the composition in a total amount ranging from 2% to 6% by weight, relative to the total weight of the composition.

34. The composition according to claim 1, further comprising at least one aqueous phase.

35. The composition according claim 34, wherein the weight ratio of water to oil is greater than or equal to 1.

36. The composition according to claim 1, further comprising at least one pulverulent phase comprising at least one pulverent chosen from pigments, fillers and nacres.

37. The composition according to claim 1, further comprising at least one wax.

38. The composition according to claim 1, further comprising at least one mineral thickener.

39. The composition according to claim 1, wherein said composition is in a form chosen from: anhydrous form; water-in-oil, oil-in-water and multiple emulsions; gels; creams; suspensions; compact form; hot-cast form; and in the form of sticks.

40. A process for making up or caring for keratin materials, comprising applying to the keratin materials at least one cosmetic composition comprising, in a cosmetically acceptable medium,
at least one continuous liquid fatty phase, comprising at least one compound of formula (I): 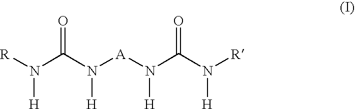
and salts and isomers thereof,
wherein:
A is chosen from groups of formula (II): 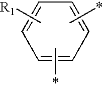
wherein R₁ is chosen from linear and branched C₁-C₄ alkyl radicals, and the asterisks, *, symbolize the points of attachment of the group A to each of the two nitrogen atoms of the rest of the compound of formula (I), and
R and R′, which may be identical or different, are chosen from:
i) the radicals of formula (III): 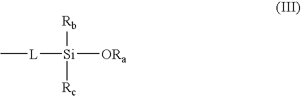
wherein:
L is chosen from a single bond, and linear, branched and cyclic, saturated and unsaturated, divalent hydrocarbon-based alkylene radicals, comprising 1 to 18 carbon atoms, and optionally comprising 1 to 4 heteroatoms chosen from N, O and S;
Rₐ is chosen from:
a) linear, branched and cyclic, saturated and unsaturated, hydrocarbon-based alkyl radicals, comprising 1 to 18 carbon atoms, optionally comprising 1 to 8 heteroatoms chosen from N, O, Si and S; and
b) silicone radicals of formula: 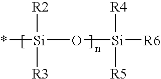
wherein n ranges from 0 to 100;
and wherein R₂ to R₆ are, independently of each other, chosen from linear and branched hydrocarbon-based alkyl radicals comprising 1 to 12 carbon atoms, and optionally comprising 1 to 4 heteroatoms;
R_{b} and R_{c} are, independently of each other, chosen from:
a) linear, branched and cyclic, saturated and unsaturated hydrocarbon-based alkyl radicals, comprising 1 to 18 carbon atoms, and optionally comprising 1 to 4 heteroatoms chosen from N, O, Si and S;
b) the radicals of formula: 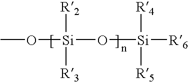
wherein n ranges from 0 to 100;
and R′₂ to R′₆ are, independently of each other, chosen from linear and branched hydrocarbon-based alkyl radicals, comprising 1 to 12 carbon atoms, and optionally comprising 1 to 4 heteroatoms; and
ii) linear, branched and cyclic, saturated and unsaturated, C1-C30 alkyl radicals, optionally comprising 1 to 3 heteroatoms chosen from O, S, F and N;
wherein at least one of the radicals R and R′ is chosen from those of formula (III); and at least one oil with a solubility parameter δₐ ranging from 0 to 7.00 (J/cm³)^{1/2}.

41. A method of obtaining a makeup result that is at least one of smooth via leveling of the skin relief, matte appearance, and soft to the touch, comprising applying to the skin, at least one cosmetic composition comprising, in a cosmetically acceptable medium, at least one continuous liquid fatty phase, comprising at least one compound of formula (I): 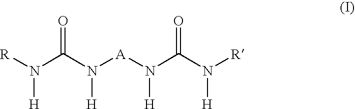
and salts and isomers thereof,
wherein:
A is chosen from groups of formula (II): 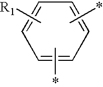
wherein R₁ is chosen from linear and branched C₁-C₄ alkyl radicals, and the asterisks, *, symbolize the points of attachment of the group A to each of the two nitrogen atoms of the rest of the compound of formula (I), and
R and R′, which may be identical or different, are chosen from:
i) the radicals of formula (III): 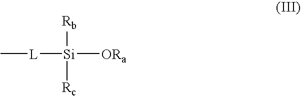
wherein:
L is chosen from a single bond, and linear, branched and cyclic, saturated and unsaturated, divalent hydrocarbon-based alkylene radicals, comprising 1 to 18 carbon atoms, and optionally comprising 1 to 4 heteroatoms chosen from N, O and S;
Rₐ is chosen from:
a) linear, branched and cyclic, saturated and unsaturated, hydrocarbon-based alkyl radicals, comprising 1 to 18 carbon atoms, optionally comprising 1 to 8 heteroatoms chosen from N, O, Si and S; and
b) silicone radicals of formula: 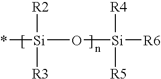
wherein n ranges from 0 to 100;
and wherein R₂ to R₆ are, independently of each other, chosen from linear and branched hydrocarbon-based alkyl radicals comprising 1 to 12 carbon atoms, and optionally comprising 1 to 4 heteroatoms;
R_{b} and R_{c} are, independently of each other, chosen from:
a) linear, branched and cyclic, saturated and unsaturated hydrocarbon-based alkyl radicals, comprising 1 to 18 carbon atoms, and optionally comprising 1 to 4 heteroatoms chosen from N, O, Si and S;
b) the radicals of formula: 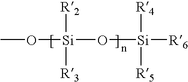
wherein n ranges from 0 to 100;
and R′₂ to R′₆ are, independently of each other, chosen from linear and branched hydrocarbon-based alkyl radicals, comprising 1 to 12 carbon atoms, and optionally comprising 1 to 4 heteroatoms; and
ii) linear, branched and cyclic, saturated and unsaturated, C₁-C₃₀ alkyl radicals, optionally comprising 1 to 3 heteroatoms chosen from O, S, F and N;
wherein at least one of the radicals R and R′ is chosen from those of formula (III); and at least one oil with a solubility parameter δₐ ranging from 0 to 7.00 (J/cm³)^{1/2}.
